SEQUENCE LISTING (15 sequences)

SEQ ID NO: 1 - protein, 95 aa, synthetic construct
MKDKQDEEQRTRRMMRTKMRMRRMRRTRRKMRRKMSPARPRTSCREACLQGWTEAAYEEAEDNCRRMMRTKAAYVLNYGVCFCAAYFCGDENILV

SEQ ID NO: 2 - DNA, 285 bp, synthetic construct
  source 1..285: mol_type = other DNA
atgaaggacaaacaggacgaggaacagagaacaaggcggatgatgagaaccaagatgaggatgagacggatgcgcagaacacggaggaaaatgagaaggaagatgtctcctgccagacctaggaccagctgtagagaagcttgtctgcagggatggacagaagccgcttacgaagaggccgaagacaactgtcggagaatgatgcggacaaaagctgcctacgtgctgaattatggcgtgtgtttttgcgccgcatacttttgtggcgacgagaacatcctggtg

SEQ ID NO: 3 - protein, 54 aa, synthetic construct
MKDKQDEEQRTRRMMRTKMRMRRMRRTRRKMRRKMSPARPRTSCREACLQGWTE

SEQ ID NO: 4 - protein, 14 aa, synthetic construct
EEAEDNCRRMMRTK

SEQ ID NO: 5 - protein, 9 aa, synthetic construct
VLNYGVCFC

SEQ ID NO: 6 - protein, 9 aa, synthetic construct
FCGDENILV

SEQ ID NO: 7 - skipped

SEQ ID NO: 8 - protein, 116 aa, synthetic construct
MACPGFLWALVISTCLEFSMAMKDKQDEEQRTRRMMRTKMRMRRMRRTRRKMRRKMSPARPRTSCREACLQGWTEAAYEEAEDNCRRMMRTKAAYVLNYGVCFCAAYFCGDENILV

SEQ ID NO: 9 - DNA, 348 bp, synthetic construct
  source 1..348: mol_type = other DNA
atggcttgtcctggctttctgtgggccctggtgatcagcacatgtctggagttctctatggctatgaaggacaaacaggacgaggaacagagaacaaggcggatgatgagaaccaagatgaggatgagacggatgcgcagaacacggaggaaaatgagaaggaagatgtctcctgccagacctaggaccagctgtagagaagcttgtctgcagggatggacagaagccgcttacgaagaggccgaagacaactgtcggagaatgatgcggacaaaagctgcctacgtgctgaattatggcgtgtgtttttgcgccgcatacttttgtggcgacgagaacatcctggtg

SEQ ID NO: 10 - protein, 21 aa, synthetic construct
MACPGFLWALVISTCLEFSMA

SEQ ID NO: 11 - DNA, 63 bp, synthetic construct
  source 1..63: mol_type = other DNA
atggcttgtcctggctttctgtgggccctggtgatcagcacatgtctggagttctctatggct

SEQ ID NO: 12 - protein, 123 aa, synthetic construct
MGQKEQIHTLQKNSERMSKQLTRSSQAVMKDKQDEEQRTRRMMRTKMRMRRMRRTRRKMRRKMSPARPRTSCREACLQGWTEAAYEEAEDNCRRMMRTKAAYVLNYGVCFCAAYFCGDENILV

SEQ ID NO: 13 - DNA, 369 bp, synthetic construct
  source 1..369: mol_type = other DNA
atgggccagaaggaacagattcatacgcttcagaaaaattctgaacgaatgtcaaagcaattgacacgaagttctcaggcagtaatgaaggacaaacaagacgaagaacaacgaactaggcggatgatgaggactaagatgaggatgcggaggatgagacggacgcgacgcaagatgcgccggaaaatgtctcccgcccggccaaggacgtcttgtcgggaagcctgtttgcagggctggaccgaagcagcttacgaagaagcagaagacaattgtcggcgaatgatgagaacgaaggctgcttacgtgcttaactatggagtgtgcttctgcgctgcctatttctgcggagatgagaacattctggtg

SEQ ID NO: 14 - protein, 28 aa, synthetic construct
MGQKEQIHTLQKNSERMSKQLTRSSQAV

SEQ ID NO: 15 - DNA, 84 bp, synthetic construct
  source 1..84: mol_type = other DNA
atgggccagaaggaacagattcatacgcttcagaaaaattctgaacgaatgtcaaagcaattgacacgaagttctcaggcagta

(19) United States
(12) Patent Application Publication
(10) Pub. No.: US 2023/0285548 A1
(43) Pub. Date: Sep. 14, 2023

(54) VACCINES BASED ON MUTANT CALR AND JAK2 AND THEIR USES

(71) Applicant: Janssen Biotech, Inc., Horsham, PA (US)

(72) Inventors: Patrick WILKINSON, Collegeville, PA (US); Manuel Alejandro Sepulveda, West Windsor, NJ (US); Charles George Drake, New Hope, PA (US); Derick Siegel, Telford, PA (US)

(21) Appl. No.: 18/080,639

(22) Filed: Dec. 13, 2022

Related U.S. Application Data
(60) Provisional application No. 63/317,143, filed on Mar. 7, 2022.
(60) Provisional application No. 63/290,156, filed on Dec. 16, 2021.

(51) Int. Cl. A61K 39/235; A61K 39/285; A61K 39/395; A61K 45/06

(52) U.S. Cl. CPC A61K 39/235; A61K 39/285; A61K 39/3955; A61K 45/06; A61K 2039/505

(57) ABSTRACT

Disclosed herein are methods of treating or preventing a myeloproliferative disease, a cancer, or a cardiovascular disease, and methods of inducing an immune response, in a subject having a JAK2V617F substitution and/or a CALR exon 9 mutation.

### CROSS REFERENCE TO RELATED APPLICATIONS

This application claims priority to U.S. Provisional Application Serial No. 63/317,143, filed on Mar. 7, 2022, and U.S, Provisional Application Serial No. 63/290,156, filed on Dec. 16, 2021, the entire content of each of which are incorporated herein by reference in their entirety.

### SEQUENCE LISTING

The contents of the electronic sequence listing (JBI6596USNP1_Sequence Listing.xml; Size: 14,901 bytes; and Date of Creation: Nov. 22, 2022) is herein incorporated by reference in its entirety.

### BACKGROUND

The classical myeloproliferative neoplasms (MPNs), also called BCR-ABL-MPNs, are the most frequent diseases among the myeloproliferative disorders. MPNs are characterized by excessive production of terminally differentiated blood cells that are fully functional. Classical MPNs have been classified into three entities: polycythemia vera (PV), essential thrombocythemia (ET), and primary myelofibrosis (PMF), which have frequent disease-related complications, such as venous and arterial thrombosis, hemorrhages, and transformation to acute myeloid leukemia (AML). All MPN entities arise from a single somatically mutated hematopoietic stem cell (HSC) that clonally expands and gives rise to virtually all myeloid cells, and B and NK cells. The clonal expansion of the MPN HSC is accompanied by single- or multi-lineage hyperplasia.

More than 50% of patients with MPNs harbor the JAK2V617F mutation, which is caused by a guanine (G) to thymine (T) somatic mutation at nucleotide 1849, in Exon 14 of JAK2, resulting in the substitution of valine to phenylalanine at codon 617 in the pseudokinase domain. In addition, mutations in exon 9 of the calreticulin (CALR) gene are found in approximately 60% of patients with JAK2 wild type essential thrombocytemia (ET) or primary myelofibrosis (PMF).

MPN patients have high symptom burden, life-threatening complications, and risk of progression to acute leukemia while also having limited treatment options. MPN patient treatments are best divided into the categories of observation, medical therapies, and allogeneic stem cell transplantation (allo-SCT). Medical therapies themselves fall into the categories of cytoreductive agents, single-agent JAK inhibitors, and the immunomodulatory agent interferon α (IFNα). The current standard of care, and only approved therapeutic, specifically for patients with MPN is the small-molecule JAK½ inhibitor JAKAFI® (ruxolitinib). Efficacy of JAKAFI® was established in the COMFORT-I and COMFORT-II studies and showed significant reduction in spleen size as the primary endpoint. JAKAFI^{®}, however, was discontinued due to loss of response, disease progression, and treatment-related adverse events in about 50% of the patients at 3 years and 75% of the patients at 5 years. JAKAFI® (ruxolitinib) therapy has also been associated with increased risk for aggressive B-cell lymphoma in myelofibrosis (MF) patients. Indeed, in a study of 107 MF patients that discontinued JAKAFI® (ruxolitinib) treatment, the medium overall survival was just 14 months. Although there is a subset of patients that may derive a survival benefit with JAKAFI® (ruxolitinib) use, the majority of MPN patients continue to progress in their disease.

The JAK2V617F mutation and mutations in exon 9 of CALR have also been identified in other cancers and cardiovascular diseases.

### SUMMARY

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject a treatment regimen comprising: two or more vaccines comprising a great ape adenovirus serotype 20 (GAd20) virus that, in turn, comprises a nucleotide sequence encoding the amino acid sequence of SEQ ID NO: 1, and one or more vaccines comprising a Modified Vaccinia Ankara (MVA) virus that, in turn, comprises a nucleotide sequence encoding the amino acid sequence of SEQ ID NO: 1, to thereby treat or prevent the myeloproliferative disease, cancer, or cardiovascular disease, or induce the immune response.

Also provided are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject: a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1 × 10⁸ infectious units (IFU) of an MVA virus at about week 9, wherein the MVA virus comprises a nucleotide sequence encoding the amino acid sequence of SEQ ID NO: 1; a vaccine comprising 1 × 10¹¹ VP of the GAd20 virus at about week 15 and about week 18; a vaccine comprising 1 × 10⁸ IFU of the MVA virus at about week 24; and a vaccine comprising 1 × 10⁸ IFU of the MVA virus at about week 36, about week 48, and about week 60.

Methods of treating or preventing a myeloproliferative disease, a cancer, or a cardiovascular disease, or inducing an immune response, in a subject having a JAK2V617F substitution and/or a CALR exon 9 mutation are provided, wherein the methods comprise administering to the subject: a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1 × 10⁸ infectious units (IFU) of an MVA virus at about week 9, wherein the MVA virus comprises a nucleotide sequence encoding the amino acid sequence of SEQ ID NO: 1; a vaccine comprising 1 × 10¹¹ VP of the GAd20 virus at about week 15; a vaccine comprising 1 × 10⁸ IFU of the MVA virus at about week 24; and a vaccine comprising 1 × 10⁸ IFU of the MVA virus at about week 36, about week 48, and about week 60.

Disclosed are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject: a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1 × 10⁸ infectious units (IFU) of an MVA virus at about week 9, wherein the MVA virus comprises a nucleotide sequence encoding the amino acid sequence of SEQ ID NO: 1; a vaccine comprising 1 × 10⁸ IFU of the MVA virus at about week 15, about week 18, and about week 24; and a vaccine comprising 1 × 10⁸ IFU of the MVA virus at about week 36, about week 48, and about week 60.

Provided are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject: a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1 × 10⁸ infectious units (IFU) of an MVA virus at about week 9, wherein the MVA virus comprises a nucleotide sequence encoding the amino acid sequence of SEQ ID NO: 1; a vaccine comprising 1 × 10⁸ IFU of the MVA virus at about week 15 and about week 24; and a vaccine comprising 1 × 10⁸ IFU of the MVA virus at about week 36, about week 48, and about week 60.

Methods of treating or preventing a myeloproliferative disease, a cancer, or a cardiovascular disease, or inducing an immune response, in a subject having a JAK2V617F substitution and/or a CALR exon 9 mutation are disclosed, wherein the methods comprise administering to the subject: 1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-CTLA4 antibody and a vaccine comprising 1 × 10¹¹ VP of the GAd20 virus at about week 15 and about week 18; 1 mg/kg to 3 mg/kg of an anti-CTLA4 antibody and a vaccine comprising 1 × 10⁸ IFU of the MVA virus at about week 24; and 1 mg/kg to 3 mg/kg of an anti-CTLA4 antibody and a vaccine comprising 1 × 10⁸ IFU of the MVA virus at about week 36, about week 48, and about week 60.

Methods of treating or preventing a myeloproliferative disease, a cancer, or a cardiovascular disease, or inducing an immune response, in a subject having a JAK2V617F substitution and/or a CALR exon 9 mutation are disclosed, wherein the methods comprise administering to the subject: 1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-PD1 antibody and a vaccine comprising 1 × 10¹¹ VP of the GAd20 virus at about week 15 and about week 18; 1 mg/kg to 3 mg/kg of an anti-PD1 antibody and a vaccine comprising 1 × 10⁸ IFU of the MVA virus at about week 24; and 1 mg/kg to 3 mg/kg of an anti-PD1 antibody and a vaccine comprising 1 × 10⁸ IFU of the MVA virus at about week 36, about week 48, and about week 60.

Provided herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject: 1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-CTLA4 antibody and a vaccine comprising 1 × 10¹¹ VP of the GAd20 virus at about week 15; 1 mg/kg to 3 mg/kg of an anti-CTLA4 antibody and a vaccine comprising 1 × 10⁸ IFU of the MVA virus at about week 24; and 1 mg/kg to 3 mg/kg of an anti-CTLA4 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject: 1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-PD1 antibody and a vaccine comprising 1 × 10¹¹ VP of the GAd20 virus at about week 15; 1 mg/kg to 3 mg/kg of an anti-PD1 antibody and a vaccine comprising 1 × 10⁸ IFU of the MVA virus at about week 24; and 1 mg/kg to 3 mg/kg of an anti-PD1 antibody and a vaccine comprising 1 × 10⁸ IFU of the MVA virus at about week 36, about week 48, and about week 60.

Methods of treating or preventing a myeloproliferative disease, a cancer, or a cardiovascular disease, or inducing an immune response, in a subject having a JAK2V617F substitution and/or a CALR exon 9 mutation are disclosed, wherein the methods comprise administering to the subject: 1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-CTLA4 antibody and a vaccine comprising 1 × 10⁸ IFU of the MVA virus at about week 15, about week 18, and about week 24; and 1 mg/kg to 3 mg/kg of an anti-CTLA4 antibody and a vaccine comprising 1 × 10⁸ IFU of the MVA virus at about week 36, about week 48, and about week 60.

Provided are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject: 1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-PD1 antibody and a vaccine comprising 1 × 10⁸ IFU of the MVA virus at about week 15, about week 18, and about week 24; and 1 mg/kg to 3 mg/kg of an anti-PD1 antibody and a vaccine comprising 1 × 10⁸ IFU of the MVA virus at about week 36, about week 48, and about week 60.

Methods of treating or preventing a myeloproliferative disease, a cancer, or a cardiovascular disease, or inducing an immune response, in a subject having a JAK2V617F substitution and/or a CALR exon 9 mutation are disclosed, wherein the methods comprise administering to the subject: 1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-CTLA4 antibody and a vaccine comprising 1 × 10⁸ IFU of the MVA virus at about week 15 and about week 24; and 1 mg/kg to 3 mg/kg of an anti-CTLA4 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject: 1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1; 1 mg/kg to 3 mg/kg of an anti-PD1 antibody and a vaccine comprising 1 × 10⁸ IFU of the MVA virus at about week 15 and about week 24; and 1 mg/kg to 3 mg/kg of an anti-PD1 antibody and a vaccine comprising 1 × 10⁸ IFU of the MVA virus at about week 36, about week 48, and about week 60.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1 illustrates an exemplary dosing schedule.

FIG. 2 illustrates the number of cynomolgus monkeys exhibiting CALR specific IFNy+ T cells (SFU/10⁶ cells) (responders) at weeks 0, 3, and 5 after receiving 1 × 10¹¹ VP of GAd20-HCalJ-9.9 + 3 mg/kg ipilimumab (Group 1 or Group 2).

FIG. 3 illustrates the number of cynomolgus monkeys exhibiting CALR specific IFNy+ T cells (SFU/10⁶ cells) (responders) at day 64 and 78 following administration of MVA-HCalJ-9.9 (n=20).

FIG. 4A and FIG. 4B illustrate the number of cynomolgus monkeys exhibiting CALR peptide pool specific IFNy+ T cells (SFU/10⁶ cells) (responders) after receiving the indicated dosing regimen (Group 1 or Group 2, as further described in Table 2 herein). 2nd cycle of GAd20/GAd20/MVA maintains antigen specific T cell response through day 183.

FIG. 5 illustrates a schematic overview of the VAC85135MPN1001 Study.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the methods as described herein. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” is used to encompass variations of ± 10% or less, variations of ± 5% or less, variations of ± 1% or less, variations of ± 0.5% or less, or variations of ± 0.1% or less from the specified value

The term “comprising” is intended to include examples encompassed by the terms “consisting essentially of” and “consisting of”; similarly, the term “consisting essentially of” is intended to include examples encompassed by the term “consisting of.”

“Administered with” means that two or more therapeutics (such as a virus and an antibody) can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms of a myeloproliferative disease, a cancer, or a cardiovascular disease, eliminating symptoms and/or the underlying cause of the symptoms of a myeloproliferative disease, a cancer, or a cardiovascular disease, reducing the frequency or likelihood of symptoms of a myeloproliferative disease, a cancer, or a cardiovascular disease and/or their underlying cause, and improving or remediating damage caused, directly or indirectly, by a myeloproliferative disease, a cancer, or a cardiovascular disease. Treatment also includes prolonging survival as compared to the expected survival of a subject not receiving treatment. Subjects to be treated include those that have a myeloproliferative disease, a cancer, or a cardiovascular disease as well as those prone to have, or those in which, a myeloproliferative disease, a cancer, or a cardiovascular disease is to be prevented.

Disclosed herein are methods of treating or preventing a myeloproliferative disease, a cancer, or a cardiovascular disease, and methods of inducing an immune response, in a subject having a JAK2V617F substitution and/or a CALR exon 9 mutation.

A guanine (G) to thymine (T) somatic mutation at nucleotide 1849 in exon 14 of JAK2 results in the substitution of valine to phenylalanine at codon 617 (JAK2V617F) in the pseudokinase domain. This mutation can be found in around 70% of myeloproliferative neoplasms (MPNs): 95% of polycythemia vera (PV) and 50% to 60% of ET and PMF. JAK2V617F often undergoes a transition from heterozygosity to homozygosity due to occurrence of mitotic recombination resulting in copy-neutral loss of heterozygosity along a variable size region on the short arm of Chromosome 9 (9pLOH). JAK2V617F arises in a multipotent hematopoietic progenitor, is present in all myeloid lineages, and can be also detected in lymphoid cells, mainly B and natural killer (NK) cells and more rarely and later in disease in T cells. JAK2V617F is mainly restricted to classical MPNs with the exception of refractory anemia with ring sideroblasts and thrombocytosis (RARS T). JAK2V617F has been detected at very low level (lower than 1%) in the normal population, including in a neonate. It is one of the most frequent mutations found in the clonal hematopoiesis associated with aging (clonal hematopoiesis of indeterminate potential). The presence of JAK2V71F mutations leads to constitutive activation of signal transducer and activator of transcription (STAT) signaling leading to increased cell proliferation, activation, and autocrine/paracrine release. JAK2V617F mutation has also been identified in patients with cardiovascular indications.

Frameshift mutations in exon 9 of the CALR gene were identified in essential thrombocythemia (ET) and primary myelofibrosis (PMF) patients that were negative for the JAK2V617F mutation and for mutations in the thrombopoietin receptor (MPL) gene. Over 50 frameshift mutations were identified, with >85% leading to an identical 44-amino-acid-mutant C terminal tail. Mutation of the C terminal tail removes a KDEL motif leading to loss of endoplasmic reticulum (ER) retention and translocation to the cell surface membrane. Additionally, the mutant version of CALR has a positively charged C terminal tail that disrupts Ca2+ binding and that limits canonical function. The two most frequent CALR mutations correspond to a 52 bp deletion (p.L367fs*46), also called Type 1, and a 5 bp insertion (p.K385fs*47), also called Type 2. There are great differences in the frequency between Type 1 and Type 2 mutations in ET and PMF: in ET, Type 1 and Type 2 mutations are closely distributed (55% versus 35%), whereas in PMF, Type 1 are largely predominant (75% versus 15%). Altogether, these results indicate that mutant CALR is an oncogenic driver and that CALRmut induces transformation through the MPL-JAK2-STAT signaling pathway.

The disclosed methods can comprise administering to the subject a treatment regimen comprising:
two or more vaccines comprising a great ape adenovirus serotype 20 (GAd20) virus that, in turn, comprises a nucleotide sequence encoding the amino acid sequence of SEQ ID NO: 1 and
one or more vaccines comprising a Modified Vaccinia Ankara (MVA) virus that, in turn, comprises a nucleotide sequence encoding the amino acid sequence of SEQ ID NO: 1,
to thereby treat or prevent the myeloproliferative disease, cancer, or cardiovascular disease, or induce the immune response.

SEQ ID NO: 1 comprises the amino acid sequence of two CALR epitopes [epitope 1:

MKDKQDEEQRTRRMMRTKMRMRRMRRTRRKMRRKMSPARPRTSCREACLQGWTE (SEQ ID NO: 3); and epitope 2: EEAEDNCRRMMRTK (SEQ ID NO: 4)], two JAK2 epitopes [epitope 1: VLNYGVCFC (SEQ ID NO: 5); and epitope 2: FCGDENILV (SEQ ID NO: 6)], and AAY linkers (SEQ ID NO: 7) separating each epitope. The AAY linkers promote proteasomal cleavage of the peptide. Vaccines comprising a nucleotide sequence that encodes the amino acid sequence of SEQ ID NO: 1 induce immune responses to the JAK2V617F substitution and/or a CALR exon 9 mutation.

GAd20 is an adenovirus that infects gorilla (Gorilla), and can be isolated from stool samples of the gorilla. The GAd20 can be engineered to comprise at least one functional deletion or a complete removal of a gene product that is essential for viral replication, such as one or more of the adenoviral regions E1, E2 and E4, therefore rendering the adenovirus to be incapable of replication. The deletion of the El region may comprise deletion of EIA, EIB 55 K or EIB 21 K, or any combination thereof. Replication deficient adenoviruses are propagated by providing the proteins encoded by the deleted region(s) in trans by the producer cell by utilizing helper plasmids or engineering the producer cell to express the required proteins. Adenovirus vectors may also have a deletion in the E3 region, which is dispensable for replication, and hence such a deletion does not have to be complemented. The GAd20 of the disclosure may comprise a functional deletion or a complete removal of the E1 region and at least part of the E3 region. The GAd20 may further comprise a functional deletion or a complete removal of the E4 region and/or the E2 region. Suitable producer cells that can be utilized are human retina cells immortalized by El, e.g. 911 or PER.C6 cells (see, e.g., U.S. Pat. No. 5,994,128), El-transformed amniocytes (See, e.g., EP 1230354), E1-transformed A549 cells (see e.g. Int. Pat. Publ. No. WO1998/39411, U.S. Pat. No. 5,891,690). The nucleotide sequence encoding the amino acid sequence of SEQ ID NO: 1 may be inserted into a site or region (insertion region) in the viral genome that does not affect virus viability of the resultant recombinant virus. The nucleotide sequence encoding the amino acid sequence of SEQ ID NO: 1 may be inserted into the deleted E1 region in parallel (transcribed 5′ to 3′) or anti-parallel (transcribed in a 3′ to 5′ direction relative to the vector backbone) orientation. In addition, appropriate transcriptional regulatory elements that are capable of directing expression of the nucleotide sequence encoding the amino acid sequence of SEQ ID NO: 1 in the mammalian host cells that the virus is being prepared for use may be operatively linked to the nucleotide sequence. “Operatively linked” sequences include both expression control sequences that are contiguous with the nucleic acid sequences that they regulate and regulatory sequences that act in trans, or at a distance to control the regulated nucleic acid sequence.

Recombinant GAd20 particles may be prepared and propagated according to any conventional technique in the field of the art (e.g., Int. Pat. Publ. No. WO1996/17070) using a complementation cell line or a helper virus, which supplies in trans the missing viral genes necessary for viral replication. The cell lines 293 (Graham et al., 1977, J. Gen. Virol. 36: 59-72), PER.C6 (see e.g. U.S. Pat. No. 5,994,128), E1 A549 and 911 are commonly used to complement El deletions. Other cell lines have been engineered to complement defective vectors (Yeh, et al., 1996, J. Virol. 70: 559-565; Kroughak and Graham, 1995, Human Gene Ther. 6: 1575-1586; Wang, et al., 1995, Gene Ther. 2: 775-783; Lusky, et al., 1998, J. Virol. 72: 2022-203; EP 919627 and Int. Pat. Publ. No. WO1997/04119). The GAd20 particles may be recovered from the culture supernatant but also from the cells after lysis and optionally further purified according to standard techniques (e.g., chromatography, ultracentrifugation, as described in Int. Pat. Publ. No. WO1996/27677, Int. Pat. Publ. No. WO1998/00524, Int. Pat. Publ. No. WO1998/26048 and Int. Pat. Publ. No. WO2000/50573). The construction and methods for propagating adenoviral vectors, such as GAd20, are also described in for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913.

MVA originates from the dermal vaccinia strain Ankara (Chorioallantois vaccinia Ankara (CVA) virus) that was maintained in the Vaccination Institute, Ankara, Turkey for many years and used as the basis for vaccination of humans. However, due to the often severe post-vaccinal complications associated with vaccinia viruses (VACV), there were several attempts to generate a more attenuated, safer smallpox vaccine.

MVA has been generated by 516 serial passages on chicken embryo fibroblasts of the CVA virus (see Meyer et al., J. Gen. Virol., 72: 1031-1038 (1991) and U.S. Pat. No. 10,035,832). As a consequence of these long-term passages the resulting MVA virus deleted about 31 kilobases of its genomic sequence and, therefore, was described as highly host cell restricted to avian cells (Meyer, H. et al.,; Meisinger-Henschel et al., J. Gen. Virol. 88, 3249-3259, 2007). Comparison of the MVA genome to its parent, CVA, revealed 6 major deletions of genomic DNA (deletion I, II, III, IV, V, and VI), totaling 31,000 basepairs. (Meyer et al., J. Gen. Virol. 72:1031-8 (1991)). It was shown in a variety of animal models that the resulting MVA was significantly avirulent (Mayr, A. & Danner, K. Vaccination against pox diseases under immunosuppressive conditions, Dev. Biol. Stand. 41: 225-34, 1978). Being that many passages were used to attenuate MVA, there are a number of different strains or isolates, depending on the passage number in CEF cells, such as MVA 476 MG/14/78, MVA-571, MVA-572, MVA-574, MVA-575 and MVA-BN. MVA 476 MG/14/78 is described for example in Int. Pat. Publ. No. WO2019/115816A1. MVA-572 strain was deposited at the European Collection of Animal Cell Cultures (“ECACC”), Health Protection Agency, Microbiology Services, Porton Down, Salisbury SP4 0JG, United Kingdom (“UK”), under the deposit number ECACC 94012707 on Jan. 27, 1994. MVA-575 strain was deposited at the ECACC under deposit number ECACC 00120707 on Dec. 7, 2000; MVA-Bavarian Nordic (“MVA-BN”) strain was deposited at the ECACC under deposit number V00080038 on Aug. 30, 2000. The genome sequences of MVA-BN and MVA-572 are available at GenBank (Accession numbers DQ983238 and DQ983237, respectively). The genome sequences of other MVA strains can be obtained using standard sequencing methods.

The MVA can be derived from any MVA strain or further derivatives of the MVA strain. A further exemplary MVA strain is deposit VR-1508, deposited at the American Type Culture collection (ATCC), Manassas, Va. 20108, USA. “Derivatives” of MVA refer to viruses exhibiting essentially the same characteristics as the parent MVA, but exhibiting differences in one or more parts of their genomes. In some embodiments, the MVA vector is derived from MVA 476 MG/14/78 . In some embodiments, the MVA vector is derived from MVA-571. In some embodiments, the MVA vector is derived from MVA-572. In some embodiments, the MVA vector is derived from MVA-574. In some embodiments, the MVA vector is derived from MVA-575. In some embodiments, the MVA vector is derived from MVA-BN.

The nucleotide sequence encoding the amino acid sequence of SEQ ID NO: 1 may be inserted into a site or region (insertion region) in the MVA viral genome that does not affect virus viability of the resultant recombinant virus. Such regions can be readily identified by testing segments of virus DNA for regions that allow recombinant formation without seriously affecting virus viability of the recombinant virus. The thymidine kinase (TK) gene is an insertion region that may be used and is present in many viruses, such as in all examined poxvirus genomes. Additionally, MVA contains 6 natural deletion sites, each of which may be used as insertion sites (e.g. deletion I, II, III, IV, V, and VI; see e.g. U.S. Pat. No. 5,185,146 and U.S. Pat. No. 6.440,442). One or more intergenic regions (IGR) of the MVA may also be used as an insertion site, such as IGRs IGR07/08, IGR 44/45, IGR 64/65, IGR 88/89, IGR 136/137, and IGR 148/149 (see e.g. U.S. Pat. Publ. No. 2018/0064803). Additional suitable insertion sites are described in Int. Pat. Publ. No. WO2005/048957.

MVA virus can be prepared as previously described (Piccini, et al., 1987, Methods of Enzymology 153: 545-563; U.S. Pat. No. 4,769,330; U.S. Pat. No. 4,772,848; U.S. Pat. No. 4,603,112; U.S. Pat. No. 5,100,587 and U.S. Pat. No. 5,179,993). In an exemplary method, the DNA sequence to be inserted into the viral genome can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences. MVA particles may be recovered from the culture supernatant or from the cultured cells after a lysis step (e.g., chemical lysis, freezing/thawing, osmotic shock, sonication and the like). Consecutive rounds of plaque purification can be used to remove contaminating wild type virus. Viral particles can then be purified using the techniques known in the art (e.g., chromatographic methods or ultracentrifugation on cesium chloride or sucrose gradients).

The methods can further comprise administering one or more vaccines comprising the GAd20 virus, one or more vaccines comprising the MVA virus, or one or more vaccines comprising the GAd20 virus and one or more vaccines comprising the MVA virus. In some embodiments, the methods can further comprise administering the treatment regimen two or more times. After the initial treatment regimen, for example, the methods can comprise administering two vaccines comprising the GAd20 virus and one vaccine comprising the MVA virus. In some embodiments, the methods can further comprise administering one vaccine comprising the GAd20 virus and one vaccine comprising the MVA virus. In some embodiments, the methods can further comprise administering one or more vaccines comprising the MVA virus. The methods can further comprise, for example, administering three vaccines comprising the MVA virus. The methods can further comprise, for example, administering two vaccines comprising the MVA virus.

Suitable amounts of the GAd20 virus can comprise about 1 × 10⁹ viral particles (VP) to about 1 × 10¹³ VP of the GAd20 virus.

Suitable amounts of the MVA virus can comprise about 1 × 10⁶ infectious units (IFU) to about 1 × 10¹⁰ IFU of the MVA virus.

The methods can comprise administering a vaccine comprising the GAd20 virus at week 0 and about week 3 and administering a vaccine comprising the MVA virus at about week 9, and:
Administering a vaccine comprising the GAd20 virus at about week 15 and about week 18 and administering a vaccine comprising the MVA virus at about week 24;
Administering a vaccine comprising the GAd20 virus at about week 15 and administering a vaccine comprising the MVA virus at about week 24;
Administering a vaccine comprising the MVA virus at about week 15, about week 18, and about week 24; or
Administering a vaccine comprising the MVA virus at about week 15 and about week 24.
In some embodiments, the methods further comprise administering a vaccine comprising the MVA virus at about week 36, about week 48, and about week 60. The methods can further comprise administering one or more further vaccines comprising the MVA virus.

The methods can comprise administering a vaccine comprising 1 × 10¹¹ VP of the GAd20 virus at week 0 and about week 3 and administering a vaccine comprising 1 × 10⁸ IFU of the MVA virus at about week 9, and:
Administering a vaccine comprising 1 × 10¹¹ VP of the GAd20 virus at about week 15 and about week 18 and administering a vaccine comprising 1 × 10⁸ IFU of the MVA virus at about week 24;
Administering a vaccine comprising 1 × 10¹¹ VP of the GAd20 virus at about week 15 and administering a vaccine comprising 1 × 10⁸ IFU of the MVA virus at about week 24;
Administering a vaccine comprising 1 × 10⁸ IFU of the MVA virus at about week 15, about week 18, and about week 24; or
Administering a vaccine comprising 1 × 10⁸ IFU of the MVA virus at about week 15 and about week 24.
In some embodiments, the methods further comprise administering a vaccine comprising 1 × 10⁸ IFU of the MVA virus at about week 36, about week 48, and about week 60. The methods can further comprise administering one or more further vaccines comprising 1 × 10⁸ IFU of the MVA virus.

The disclosed methods can comprise the exemplary treatment schedules provided in Table 1 below:

TABLE 1

Exemplary treatment schedules

| | Cycle 1* | | | Cycle 2 | | | Maintenance | | | | |
|---|---|---|---|---|---|---|---|---|---|---|---|
| | Dose 1 (wk0) | Dose 2 (wk3) | Dose 3 (wk9) | Dose 1 (wk15) | Dose 2 (wk18) | Dose 3 (wk24) | Dose 1 (wk36) | Dose 2 (wk48) | Dose 3 (wk60) | Dose 4 | Dose 5 |
| 1 | GAd20 | GAd20 | MVA | GAd20 | GAd20 | MVA | MVA | MVA | MVA | MVA | MVA |
| 2 | GAd20 | GAd20 | MVA | GAd20 | | MVA | MVA | MVA | MVA | MVA | MVA |
| 3 | GAd20 | GAd20 | MVA | MVA | MVA | MVA | MVA | MVA | MVA | MVA | MVA |
| 4 | GAd20 | GAd20 | MVA | MVA | | MVA | MVA | MVA | MVA | MVA | MVA |

*Cycle 1 is referred to herein as a “treatment regimen.”

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10¹¹ VP of the GAd20 virus at about week 15 and about week 18;
a vaccine comprising 1 × 10⁸ IFU of the MVA virus at about week 24; and
a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10¹¹ VP of the GAd20 virus at about week 15;
a vaccine comprising 1 × 10⁸ IFU of the MVA virus at about week 24; and
a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10⁸ IFU of the MVA virus at about week 15, about week 18, and about week 24; and
a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10⁸ IFU of the MVA virus at about week 15 and about week 24; and
a vaccine comprising 1 × 10⁸ IFU of the MVA virus at about week 36, about week 48, and about week 60.

Any of the above methods can further comprise administering an anti-CTLA4 antibody. The anti-CTLA4 antibody can be administered with the vaccines comprising the GAd20 virus, with the vaccines comprising the MVA virus, or both. Suitable amounts of the anti-CTLA4 antibody comprise about 0.5 mg/kg to about 5 mg/kg. Any antagonistic anti-CTLA4 antibody can be used in the disclosed methods. Suitable anti-CTLA4 antibodies for use in the disclosed methods include, without limitation, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, and chimeric anti-CTLA4 antibodies. Non-limiting examples of anti-CTLA4 antibodies include Ipilimumab and tremelimumab.

Any of the above methods can further comprise administering an anti-PD-1 antibody. The anti-PD-1 antibody can be administered with the vaccines comprising the GAd20 virus, with the vaccines comprising the MVA virus, or both. Suitable amounts of the anti-PD-1 antibody comprise about 0.5 mg/kg to about 5 mg/kg. Any antagonistic anti-PD-1 antibody can be used in the disclosed methods. Suitable anti-PD-1 antibodies for use in the disclosed methods include, without limitation, human anti-PD-1 antibodies, mouse anti-PD-1 antibodies, mammalian anti-PD-1 antibodies, humanized anti-PD-1 antibodies, monoclonal anti-PD-1 antibodies, polyclonal anti-PD-1 antibodies, and chimeric anti-PD-1 antibodies. Non-limiting examples of anti-PD-1 antibodies include cetrelimab, pembrolizumab, nivolumab, sintilimab, cemiplimab, toripalimab, camrelizumab, tislelizumab, dostralimab, spartalizumab, prolgolimab, balstilimab, budigalimab, sasanlimab, avelumab, atezolizumab, durvalumab, envafolimab, and iodapolimab.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10¹¹ VP of the GAd20 virus at about week 15 and about week 18;
1 mg/kg to 3 mg/kg of an anti-CTLA4 antibody and a vaccine comprising 1 × 10⁸ IFU of the MVA virus at about week 24; and
1 mg/kg to 3 mg/kg of an anti-CTLA4 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10¹¹ VP of the GAd20 virus at about week 15 and about week 18;
1 mg/kg to 3 mg/kg of an anti-PD1 antibody and a vaccine comprising 1 × 10⁸ IFU of the MVA virus at about week 24; and
1 mg/kg to 3 mg/kg of an anti-PD1 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10¹¹ VP of the GAd20 virus at about week 15;
1 mg/kg to 3 mg/kg of an anti-CTLA4 antibody and a vaccine comprising 1 × 10⁸ IFU of the MVA virus at about week 24; and
1 mg/kg to 3 mg/kg of an anti-CTLA4 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10¹¹ VP of the GAd20 virus at about week 15;
1 mg/kg to 3 mg/kg of an anti-PD1 antibody and a vaccine comprising 1 × 10⁸ IFU of the MVA virus at about week 24; and
1 mg/kg to 3 mg/kg of an anti-PD1 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10⁸ IFU of the MVA virus at about week 15, about week 18, and about week 24; and
1 mg/kg to 3 mg/kg of an anti-CTLA4 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10⁸ IFU of the MVA virus at about week 15, about week 18, and about week 24; and
1 mg/kg to 3 mg/kg of an anti-PD1 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10⁸ IFU of the MVA virus at about week 15 and about week 24; and
1 mg/kg to 3 mg/kg of an anti-CTLA4 antibody and a vaccine comprising 1 × 10⁸ IFU of the MVA virus at about week 36, about week 48, and about week 60.

Disclosed herein are methods of treating or preventing a myeloproliferative disease, a cancer, or a cardiovascular disease, or inducing an immune response, in a subject having a JAK2V617F substitution and/or a CALR exon 9 mutation, the methods comprising administering to the subject:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10⁸ IFU of the MVA virus at about week 15 and about week 24; and
1 mg/kg to 3 mg/kg of an anti-PD1 antibody and a vaccine comprising 1 × 10⁸ IFU of the MVA virus at about week 36, about week 48, and about week 60.

Each of the one or more GAd20 viruses can comprise the nucleotide sequence of SEQ ID NO: 2. In some embodiments, the nucleotide sequence further comprises an N-terminal T-cell enhancer (TCE). The TCE can comprise the HAVT20 leader seq having the amino acid sequence of SEQ ID NO: 10. The TCE can be encoded by the nucleotide sequence of SEQ ID NO: 11. The GAd20 virus can comprise a nucleotide sequence encoding the amino acid sequence of SEQ ID NO: 8 (which is referred to in the Examples as GAd20-HCalJ-9.9 or GAd20-CALR-JAK2). In some embodiments, the GAd20 virus can comprise the nucleotide sequence of SEQ ID NO: 9.

Each of the one or more MVA viruses can comprise the nucleotide sequence of SEQ ID NO: 2. In some embodiments, the nucleotide sequence further comprises an N-terminal TCE. The TCE can comprise the mandarin fish TCE having the amino acid sequence of SEQ ID NO: 14. The TCE can be encoded by the nucleotide sequence of SEQ ID NO: 15. The MVA virus can comprise a nucleotide sequence encoding the amino acid sequence of SEQ ID NO: 12 (which is referred to in the Examples as MVA-HCalJ-9.9 or MVA-CALR-JAK2). In some embodiments, the MVA virus can comprise the nucleotide sequence of SEQ ID NO: 13.

The disclosed methods can treat any myeloproliferative disease associated with a JAK2V617F substitution and/or a CALR exon 9 mutation. Exemplary myeloproliferative diseases include primary myelofibrosis (MPN), polycythemia vera (PV), essential thrombocythemia (ET), primary myelofibrosis (PMF), secondary myelofibrosis, acute myeloid leukemia (AML), secondary AML, chronic myelogenous leukemia (CML), clonal hematopoiesis of indeterminate potential (CHIP), and chronic myelomonocytic leukemia (CMML).

The disclosed methods can treat any cancer associated with a JAK2V617F substitution and/or a CALR exon 9 mutation. Exemplary cancers include lung cancer, lymphoid cancer, acute lymphoid leukemia, AML, CML, Burkitt’s lymphoma, Hodgkin’s lymphoma, plasma cell myeloma, biliary tract cancer, bladder cancer, liver cancer, pancreatic cancer, prostate cancer, skin cancer, thyroid cancer, stomach cancer, large intestine cancer, colon cancer, urinary tract cancer, central nervous system cancer, neuroblastoma, kidney cancer, breast cancer, cervical cancer, testicular cancer, and soft tissue cancer.

The disclosed methods can treat any cardiovascular disease associated with a JAK2V617F substitution and/or a CALR exon 9 mutation. Exemplary cardiovascular diseases include an acute coronary syndrome, an ischemic cerebrovascular disease, an ischemic heart disease, a thrombosis, a venous thromboembolism, a deep vein thrombosis, a pulmonary embolism, a catastrophic intra-abdominal thromboses, a peripheral arterial disease, a hypertension, a heart failure, an atrial fibrillation, a coronary heart disease, an atherosclerosis, and a clonal hematopoiesis.

In some embodiments, the methods comprise screening the subject for the presence of a mutation in CALR and/or JAK2 prior to treating the myeloproliferative disease, cancer, or cardiovascular disease, or inducing an immune response. In some embodiments, the methods comprise screening for the presence of a CALR mutant comprising SEQ ID NO: 3 prior to treating the myeloproliferative disease, cancer, or cardiovascular disease, or inducing an immune response. In some embodiments, the methods comprise screening for the presence of a CALR mutant comprising SEQ ID NO: 4 prior to treating the myeloproliferative disease, cancer, or cardiovascular disease, or inducing an immune response. In some embodiments, the methods comprise screening for the presence of a JAK2 mutant comprising SEQ ID NO: 5 prior to treating the myeloproliferative disease, cancer, or cardiovascular disease, or inducing an immune response. In some embodiments, the methods comprise screening for the presence of a JAK2 mutant comprising SEQ ID NO: 6 prior to treating the myeloproliferative disease, cancer, or cardiovascular disease, or inducing an immune response. In some embodiments, the methods comprise screening for the presence of one or more CALR mutants comprising SEQ ID NOs: 3 and 4, one or more JAK2 mutants comprising SEQ ID NOs: 5 and 6, or a combination of one or more CALR mutants comprising SEQ ID NOs: 3 and 4 and one or more JAK2 mutants comprising SEQ ID NOs: 5 and 6. For example, the disclosed methods of treating or preventing a myeloproliferative disease, a cancer, or a cardiovascular disease, or inducing an immune response, in a subject having a JAK2V617F substitution and/or a CALR exon 9 mutation can comprise:
screening for the presence of one or more CALR mutants comprising SEQ ID NOs: 3 and 4, one or more JAK2 mutants comprising SEQ ID NOs: 5 and 6, or a combination of one or more CALR mutants comprising SEQ ID NOs: 3 and 4 and one or more JAK2 mutants comprising SEQ ID NOs: 5 and 6; and
if the one or more CALR mutants and/or JAK2 mutants are detected, administering to the subject a treatment regimen comprising:
   two or more vaccines comprising a great ape adenovirus serotype 20 (GAd20) virus that, in turn, comprises a nucleotide sequence encoding the amino acid sequence of SEQ ID NO: 1 and
   one or more vaccines comprising a Modified Vaccinia Ankara (MVA) virus that, in turn, comprises a nucleotide sequence encoding the amino acid sequence of SEQ ID NO: 1
to thereby treat or prevent the myeloproliferative disease, cancer, or cardiovascular disease, or induce the immune response.

The screening can comprise analyzing the presence of the mutant CALR and/or JAK2 protein or analyzing the presence of a nucleic acid sequence encoding the mutant CALR and/or JAK2 protein. Exemplary screening techniques include, for example, genotyping, PCR, and protein analysis.

In some embodiments, the methods disclosed herein comprise treating myeloproliferative disease in a patient who has previously received prior treatments, such as prior treatment with any JAK2 inhibitor, prior treatment with chemotherapy or immune therapy, or prior treatment with interferon-alpha (including PEGylated IFN-α).

### EXAMPLES

The following examples are provided to further describe some of the embodiments disclosed herein. The examples are intended to illustrate, not to limit, the disclosed embodiments.

### Example 1 – Immunogenicity of Gad20-hcalj-9.9 and Mva-hcalj-9.9 in Non-Human Primates (Cynomolgus Monkey)

The primary aim of the study was to determine whether vaccination of cynomolgus monkeys with a GAd20 comprising a nucleotide sequence encoding the amino acid sequence of SEQ ID NO: 8 (GAd20-HCalJ-9.9) and an MVA comprising a nucleotide sequence encoding the amino acid sequence of SEQ ID NO: 12 (MVA-HCalJ-9.9) together with ipilimumab induces mutCALR- and/or mutJAK2-specific T-cell responses that were higher in magnitude and duration than vaccination with GAd20-HCalJ-9.9 + ipilimumab in NHP. The secondary aim was to assess whether a second cycle of immunization (GAd20/GAd20/MVA or MVA/MVA/MVA) could further enhance or maintain the highest magnitude of antigen specific T cell response for the longest duration. The third aim of the study was to evaluate an interval of 3 weeks between the two GAd0 vaccinations and 6 week interval between the 2^{nd} GAd20 and MVA immunization for each immunization cycle.

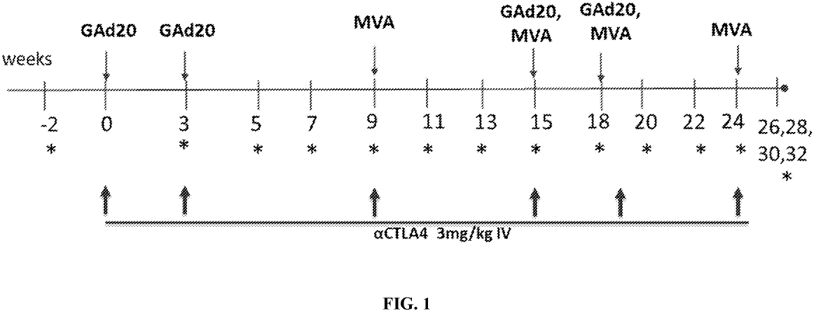

A 2-cycle dosing sequence of GAd20-HCalJ-9.9 and MVA-HCalJ-9.9 administered in combination with ipilimumab was tested in this study (see Table 2 and FIG. 1). Cycle 1 was identical for Groups 1 and 2 and consisted of 2 administrations of 1 × 10¹¹ VP GAd20-HCalJ-9.9 on Day 1 and 22, followed by administration of 1 × 10⁸ IFU MVA-HCalJ-9.9 on Day 65 by i.m. injection. Ipilimumab was co-administered at 3 mg/kg IV with each immunization on Day 1, 22, and 65. Cycle 2 vaccination was initiated 6 weeks after the last immunization in Cycle 1. Cycle 2 consisted of 2 distinct dosing schedules. Group 1 received a repeat dosing schedule identical to Cycle 1 on Days 106, 127, and 169. Group 2 Cycle 2 animals received 3 additional doses of 1 × 10⁸ IFU MVA-HCalJ-9.9 in combination with 3 mg/kg ipilimumab on Days 106, 127, and 169. GAd20-HCalJ-9.9 was injected IM into 1 limb at 1 × 10¹¹ viral particles (0.5 mL) per animal. MVA-HCalJ-9.9 was injected IM into 1 limb at 1 × 10⁸ infectious units (0.5 mL) per animal. Ipilimumab was administered IV at 0.5 mL/kg as a slow bolus over 1-3 minutes to each animal; dosing concentration was 5 mg/mL.

TABLE 2

Dosing regimens

| | Cycle 1 | | | Cycle 2 | | | |
|---|---|---|---|---|---|---|---|
| Group | Day 1 | Day 22 | Day 64 | Day 106 | Day 127 | Day 169 | No. of animals |
| 1 | GAd20-HCalJ-9.9 + ipilimumab | GAd20-HCalJ-9.9 + ipilimumab | MVA-HCalJ-9.9 + ipilimumab | GAd20-HCalJ-9.9 + ipilimumab | GAd20-HCalJ-9.9 + ipilimumab | MVA-HCalJ-9.9 + ipilimumab | 10 |
| 2 | GAd20-HCalJ-9.9 + ipilimumab | GAd20-HCalJ-9.9 + ipilimumab | MVA-HCalJ-9.9 + ipilimumab | MVA-HCalJ-9.9 + ipilimumab | MVA-HCalJ-9.9 + ipilimumab | MVA-HCalJ-9.9 + ipilimumab | 10 |

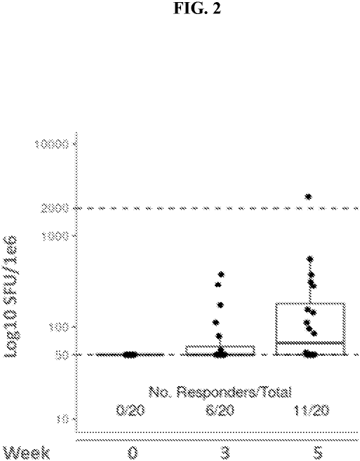

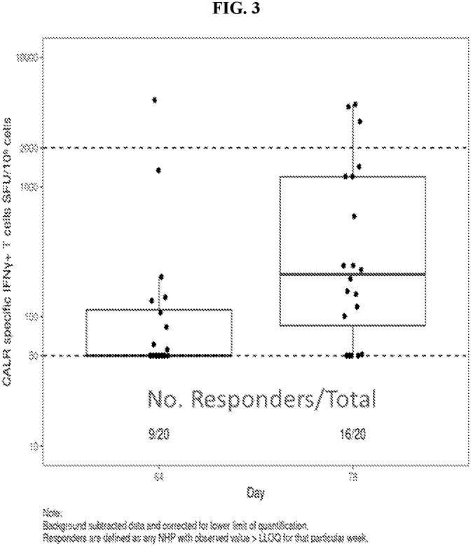

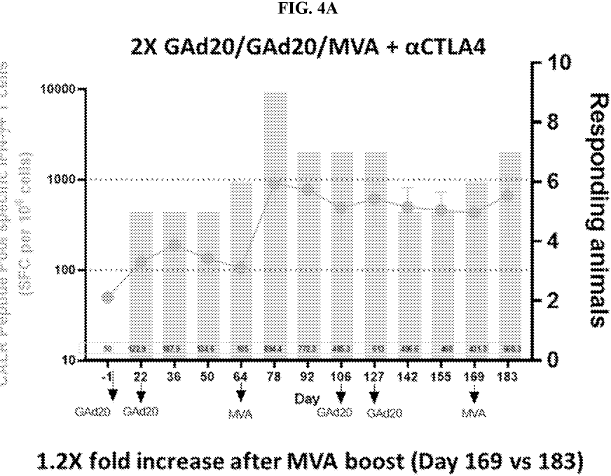

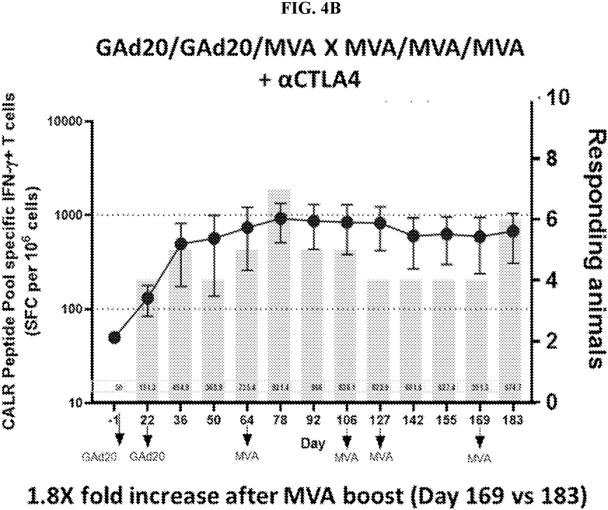

No pre-existing mutCALR T-cell responses were observed in any animal on study at Week -2 (data not shown). By week 5, 11 of 20 animals generated responses above 50 SFU/10e6 indicating animals immunized with GAd20-HCalJ-9.9 at week 0 and 3 can generate a robust antigen specific immune response that have a compatible MHC (FIG. 2). Week 11 Elispot data indicated that MVA-HCalJ9.9 can increase the magnitude of antigen specific T cell responses 2.4- fold higher than the mean responses observed immediately before MVA-HCalJ-9.9 administration (FIG. 3). Administration of a second cycle of either GAd20-HCalJ-9.9 in combination with MVA-HCalJ-9.9 or 3 immunizations of MVA-HCalJ-9.9 both maintained high levels of mutCALR specific T cell responses through 24 weeks (FIG. 4A and FIG. 4B). Animals that received a second cycle of GAd20-HCalJ-9.9 in combination with MVA-HCalJ-9.9 generated 1.8-fold increase in T cell response 2 weeks after the MVA immunization on week 24 (FIG. 4A). No negative impact on T cell response was observed with the interval of immunization that were tested in this study. Thus, GAd20-HCalJ-9.9 is immunogenic, and elicits greater IFN-γ producing mutCALR-specific T-cell responses when combined with MVA-HCalJ-9.9 and ipilimumab.

### Example 2 – Phase 1 Study - Vac85135mpn1001

### Overall Design

VAC85135MPN1001 is a Phase 1, first-in-human (FIH), open-label, multicenter study to evaluate and characterize the safety, vaccine-specific immune responses, mutCALR and JAK2V617F allele burden, and preliminary anti-tumor clinical activity of the heme vaccine administered concurrently with ipilimumab in adult participants (≥18 years of age) with myeloproliferative neoplasms (MPNs), including essential thrombocythemia (ET) that is not very low risk and myelofibrosis (MF) that is not low risk, and are mutCALR or JAK2V617F positive.

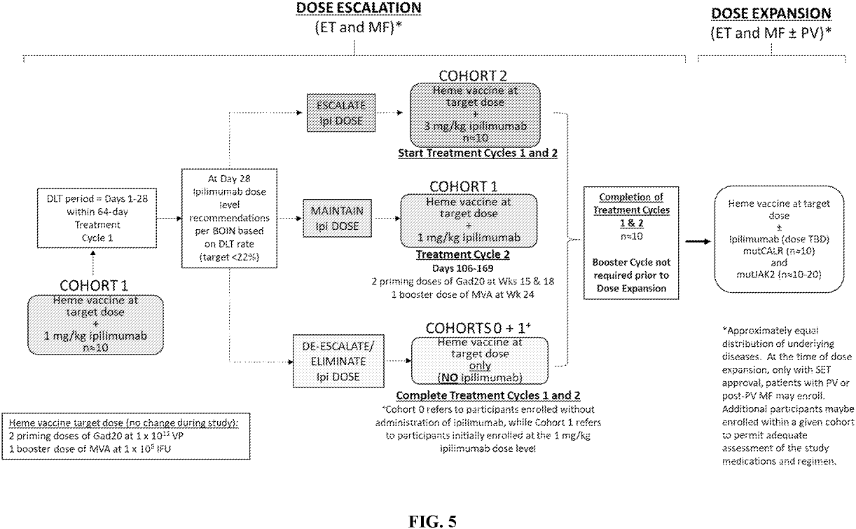

The schematic overview of the VAC85135MPN1001 study is illustrated in FIG. 5. The heme vaccine regimen encompasses up to 2 Treatment Cycles of heterologous prime-boost vaccinations followed by several booster administrations (Booster Cycle), until disease progression, intolerable toxicities, or withdrawal of consent. Treatment Cycle 1 and Treatment Cycle 2 contain 2 prime administrations with GAd20-CALR-JAK2 to prime T-cell responses, followed by 1 boost administration of MVA-CALR-JAK2 after the second priming vaccination to boost the magnitude of antigen-specific T-cell responses. After the last administration of study treatment in Treatment Cycle 2, participants will receive 3 booster vaccinations with MVA-CALR-JAK2 (Booster Cycle). GAd20-CALR-JAK2 and MVA--CALR-JAK2 will be administered via intramuscular (IM) injection. To enhance the immune response, intravenous (IV) ipilimumab (YERVOY®, anti-CTLA-4 monoclonal antibody) will be administered concurrently with each prime or boost administration of GAd20-CALR-JAK2 or MVA-CALR-JAK2, respectively. The end of treatment visit is to occur within ≤30 days (±7 days) from the last administration of booster MVA-CALR-JAK2. All participants will be monitored for an additional 12 weeks in the post-treatment safety follow-up period.

### Number of Participants

Dose Escalation: Cohort 1 will contain approximately 10 participants. If Cohort 1 is cleared for safety on the basis of the BOIN design with 22% target rate of dose-limiting toxicities (DLTs) by the end of the DLT Evaluation Period, the next dose level of ipilimumab may be tested in another cohort of approximately 10 participants (Cohort 2). Each dose level of ipilimumab, if determined to be tolerable, may be expanded. The maximum number of participants for each ipilimumab dose level will be approximately 20 to ensure that at least 10 participants have quality biomarker samples.

Dose Expansion: The Expansion cohort will contain approximately 10 evaluable participants with eligible CALR mutations and approximately 10-20 evaluable participants with the JAK2V617F mutation.

### Treatment Groups and Duration

This study will evaluate a single concentration of GAd20-CALR-JAK2 (1×10¹¹ virus particles [VP]) and MVA-CALR-JAK2 (1×10⁸ infectious units [IFU]). GAd20-CALR-JAK2 and MVA-CALR-JAK2 will be administered via IM injection. Ipilimumab will be initially administered at 1 mg/kg (Cohort 1). If ipilimumab at 1 mg/kg is tolerated, then ipilimumab at 3 mg/kg may be tested in Cohort 2. If a dose escalation cohort simultaneously enrolls >1 participant, in which a minimal interval of 7 days between the first dose of the first participant and subsequent participants is required.

### Efficacy Evaluations

Efficacy evaluations will assess the following: overall clinical response per revised response criteria by the IWG-MRT and ELN consensus report, disease burden at Week 24 and Week 48, peripheral blood mutCALR and JAK2V617F burden, bone marrow response, clinical symptoms, and time to progression or time to initiation of next therapy. In addition, the antigen-specific immune responses to the mutCALR and JAK2V617F mutations will be evaluated.

### Pharmacokinetic Evaluations

Blood samples will be collected to characterize the serum pharmacokinetics of ipilimumab after administration of the heme vaccine in combination with ipilimumab.

### Immunogenicity Evaluations

Immunogenicity evaluations will include analysis of the immunogenicity of the elements of the heme vaccine regimen (eg, vaccine vector-specific T-cell response, vaccine vector-specific antibodies) and the presence of anti-drug antibodies (ADA) to ipilimumab.

### Pharmacodynamic and Biomarker Evaluations

Blood samples will be collected for evaluation of pharmacodynamics and biomarkers after administration of the heme vaccine in combination with ipilimumab. In addition, correlation of biomarkers with clinical response or resistance to the heme vaccine administered in combination with ipilimumab will be explored.

### Safety Evaluations

The safety of the heme vaccine administered with ipilimumab will be monitored by adverse event (AE) reporting, clinical chemistry and hematology tests, electrocardiograms, vital sign measurements, physical examinations findings, and the Eastern Cooperative Oncology Group (ECOG) performance status score. The severity of AEs will be assessed using National Cancer Institute Common Terminology Criteria for Adverse Events (Version 5.0). Concomitant medication usage will be recorded.

### Statistical Methods

Dose escalation will be guided using the Bayesian Optimal Interval (BOIN) design. Dose expansion will be guided using a Beta-Binomial Bayesian model defining thresholds in dose-limiting toxicities triggering temporary halt and stop to enrollment.

### Introduction

The heme vaccine is a heterologous vaccine regimen with 2 vaccine components: a recombinant, replication-incompetent vector derived from the genome of a gorilla adenovirus serotype group C (GAd20-CALR-JAK2) and a modified-vaccinia virus Ankara vector (MVA-CALR-JAK2). Both vectors express peptide sequences derived from the common novel C-terminus of mutant versions of the calreticulin gene (mutCALR) and the valine 617 to phenylalanine mutation in the Janus kinase 2 (JAK2V617F) which are designed to elicit T-cell responses to malignant cells expressing these tumor antigens in patients with myeloproliferative neoplasms (MPNs) such as polycythemia vera (PV), essential thrombocythemia (ET), and primary myelofibrosis (PMF).

The term “study treatment” throughout the protocol, refers to the heme vaccine and ipilimumab.

### Objectives and Endpoints

Objectives and endpoints are provided in Table 3 below.

TABLE 3

Objectives and endpoints

| Objectives | Endpoints |
|---|---|
| Primary | |
| • To evaluate the safety of the heme vaccine administered with ipilimumab for the treatment of MPNs | • Incidence of dose-limiting toxicity (DLT) |
| | • Incidence of adverse events |
| Secondary | |
| • To evaluate the preliminary immunogenicity of the heme vaccine administered with ipilimumab for the treatment of MPNs | • Antigen-specific T-cell response |
| | • Overall response: per revised response criteria by the International Working Group-Myeloproliferative Neoplasms Research and Treatment (IWG-MRT) and European LeukemiaNet (ELN) consensus reportDisease response assessments at Week 24, Week 48, and EOT per modified IWG-MRT criteria |
| • To evaluate preliminary anti-tumor clinical activity of the heme vaccine administered with ipilimumab for the treatment of MPNs | • Peripheral blood mutCALR and JAK2V617F allele burden |
| | • Transfusion burden trends |
| | • Trends in patient-reported symptoms on therapy |
| | • Time to progression or time to initiation of next therapy |

Exploratory
• Analysis of vector-specific immune responses on therapy
• Pharmacokinetics of ipilimumab when co-administered with the heme vaccine
• Presence of ADAs to ipilimumab when co-administered with the heme vaccine
• Explore the relationships between, pharmacodynamics, AE profile and preliminary clinical activity of the heme vaccine administered in combination with ipilimumab
• Explore biomarkers predictive of clinical response or resistance to the heme vaccine administered in combination with ipilimumab

### Study Design

### Overall Design

VAC85135MPN1001 is a Phase 1, FIH, open-label, multicenter study to evaluate and characterize the safety, vaccine-specific immune responses, mutCALR and JAK2V617F allele burden, and preliminary anti-tumor clinical activity of the heme vaccine administered concurrently with ipilimumab in adult participants (≥18 years of age) with MPNs, including ET that is not very low risk and MF that is not low risk, and are mutCALR or JAK2V617F positive. Table 4 provides a list of the study visits and timings.

TABLE 4

Study visits and timings

| | | |
|---|---|---|
| Week Number | Visit Name | Visit Abbreviation |
| -4 to 0 | Screening | SCRN |
| 0 | Treatment Cycle 1 – Visit 1 | TC1V1 |
| 3 | Treatment Cycle 1 – Visit 2 | TC1V2 |
| 9 | Treatment Cycle 1 – Visit 3 | TC1V3 |
| 12 | Rest Period 1 | RP1 |
| 15 | Treatment Cycle 2 – Visit 1 | TC2V1 |
| 18 | Treatment Cycle 2 – Visit 2 | TC2V2 |
| 24 | Treatment Cycle 2 – Visit 3 | TC2V3 |
| 27 | Rest Period 2 | RP2 |
| 36 | Booster Cycle – Visit 1 | BCV1 |
| 48 | Booster Cycle – Visit 2 | BCV2 |
| 60 | Booster Cycle – Visit 3 | BCV3 |
| 60-64 | End Of Treatment | EOT |
| EOT+6 | Follow-up 1 | FU1 |
| EOT+12 | Follow-up 2 | FU2 |

### Dose Escalation

The first part of the study is a dose escalation phase. Only patients with essential thrombocythemia (ET) and myelofibrosis (MF) according to the 2016 WHO criteria (Arber 2016) are eligible to enroll in this phase of the study. Participants are required to have a diagnosis of ET that is not very low risk or a diagnosis of non-low risk primary myelofibrosis (PMF), post-essential thrombocythemia myelofibrosis, or prefibrotic myelofibrosis as defined in the inclusion criteria below. Analysis of a participant’s disease characteristics at screening will include cytogenetic analysis (full karyotyping or fluorescence in situ hybridization [FISH]) and molecular genetic analysis (mutational profiling). Participants will be enrolled to achieve an approximately equal number of individuals with ET and MF.

Throughout the entire study, the dose of the vaccine vectors (termed the heme vaccine target dose) administered will remain constant. The heme vaccine target dose is 1×10¹¹ virus particles of GAd20-CALR-JAK2 and 1×10⁸ infectious units (IFU) of MVA-CALR-JAK2. The heme vaccine target dose is the equivalent of the highest active dose tested in nonhuman primates (NHPs).

Ipilimumab will be administered together with Gad20-CALR-JAK2 and MVA-CALR-JAK2 to enhance the immune response. Dose escalation of ipilimumab may be explored. The initial dose of ipilimumab tested will be 1 mg/kg (Cohort 1); however, if ipilimumab at 1 mg/kg is tolerated, then a higher dose of ipilimumab at 3 mg/kg may be tested in separate cohort (Cohort 2). While the dose of ipilimumab may be altered, the heme vaccine target dose administered to all participants will remain the same throughout the entire study.

The heme vaccine regimen encompasses 2 Treatment Cycles of prime and boost intramuscular (IM) injections of GAd20-CALR-JAK2 and MVA-CALR-JAK2, respectively, followed by 3 booster IM administrations of MVA-CALR-JAK2 Booster Cycle until disease progression, intolerable toxicities, or withdrawal of consent. Treatment Cycle 1 and Treatment Cycle 2 are each approximately 9 weeks in length and contain 2 prime administrations with GAd20-CALR-JAK2 3 weeks apart to prime T-cell responses, followed by 1 boost administration of MVA-CALR-JAK2 approximately 6 weeks after the second priming vaccination to boost the magnitude of antigen-specific T-cell responses. The interval between Treatment Cycle 1 and Treatment Cycle 2 is 6 weeks. Twelve weeks after the last administration of study treatment in Treatment Cycle 2, participants will receive up to 3 booster vaccinations with MVA-CALR-JAK2 alone every 12 weeks (Booster Cycle). Cohort 1 will contain approximately 10 participants.

Ipilimumab (YERVOY®, anti-CTLA-4 monoclonal antibody) will be administered via intravenous (IV) infusion with each prime or boost IM administration of GAd20-CALR-JAK2 or MVA-CALR-JAK2, respectively. Ipilimumab will be initially administered at 1 mg/kg. The DLT evaluation period is defined as Days 1-28 in Treatment Cycle 1. If ipilimumab at 1 mg/kg is tolerated (i.e., the target DLT rate is ≤22% by the end of the DLT evaluation period, then ipilimumab at 3 mg/kg may be tested in another cohort (Cohort 2) of approximately 10 participants. To ensure participant safety in the dose escalation cohorts, a staggered dosing strategy between participants will be applied. A minimal interval of 7 days must pass from the time of the first dose of ipilimumab administered to the first participant in a dose escalation cohort and the first dose of ipilimumab administered to the next participant enrolled in that cohort. Each dose level of ipilimumab, if determined to be tolerable, may be expanded. To ensure that at least 10 participants have quality biomarker samples, approximately 10-20 participants for each ipilimumab dose level will be enrolled. Additional participants may be enrolled within a given cohort to permit adequate assessment of the study medications and regimen.

Completion of the Booster Cycle by a cohort in the Dose Escalation Phase is not required to initiate the Dose Expansion Phase of the study.

### Dose Expansion

During the dose expansion phase, patients with a diagnosis of polycythemia vera (PV) or post-polycythemia vera myelofibrosis defined by the 2016 WHO criteria will be allowed to enroll in the study. Because patients with PV almost exclusively have only the JAK2V617F mutation (in contrast to patients with ET and MF), patients with this disease will only be allowed to enroll during Dose Expansion to provide a better opportunity to balance between those participants with CALR and JAK2 mutations.

The Dose Expansion phase Cohort will contain 10-30 participants who will receive 2 Treatment Cycles of the heme vaccine at the target dose in addition to the dose of ipilimumab determined during the Dose Escalation phase. An additional cohort receiving a different dose or schedule of doses of ipilimumab may be established in the Dose Expansion phase.

### End of Treatment and Post-Treatment Follow-Up

The end of treatment visit is to occur within ≤30 days (±7 days) from the last administration of study therapy. All participants will be monitored for an additional 12 weeks (2 follow-up visits 6 weeks [±7 days] apart) in the Post-treatment safety follow-up period.

### Definition of Dose-Limiting Toxicity (DLT)

The DLT evaluation period is defined as Days 1-28 in Treatment Cycle 1 for the participants in Cohort 1.

There will be no changes in dose or dosing of GAd20-CALR-JAK2 or MVA-CALR-JAK2 in this study. Ipilimumab will be initially administered at 1 mg/kg. If ipilimumab at 1 mg/kg is tolerated (i.e., ≤22% DLT rate by the end of the DLT period), then ipilimumab at 3 mg/kg may be tested in another cohort of 10 participants (Cohort 2, (<10 participants). Each dose level of ipilimumab may be expanded if determined to be tolerable.

Toxicities will be evaluated according to NCI-CTCAE, Version 5.0. Only toxicities that occur during the DLT evaluation period will be used for the purpose of defining DLT-specific toxicities and for dose modification decisions. Evaluation Criteria for DLTs are provided in Table 5, and attribution of the DLT to one or more study therapies should be completed based on the best available clinical data.

TABLE 5

DLT Evaluation Criteria for the VAC85135MPN1001 study

| Criteria for Non-hematological Toxicity | Exceptions |
|---|---|
| Grade 3 laboratory abnormalities^{a} | Grade 3 lasting ≤72 hours^{b} (not associated with clinical complications) |
| Grade 4 laboratory abnormalities^{a} | Grade 4 lasting <24 hours^{b} (not associated with clinical complications) |
| Concurrent elevations in AST or ALT >3× ULN, total bilirubin ≥2× ULN, and alkaline phosphatase ≤2× ULN with no alternative etiology (Hy’s law) | None |
| Any other Grade 3 | Asthenia, anorexia, fever, or constipation lasting ≤6 days |
| | Nausea, vomiting, or diarrhea that has recovered in ≤4 days unless requiring tube feeding, total parenteral nutrition, or hospitalization |
| | Rash that recovers to ≤10% body surface area with medical management |
| | Local injection reaction, headache, fatigue, myalgia, or any other toxicity that improves to Grade ≤1 or baseline with outpatient treatment in ≤2 days |
| Any other Grade 4 | None |
| Any Grade 5 | None |
| Criteria for Hematological Toxicity | |
| Grade 2 immune-related thrombocytopenia, hemolytic anemia, or neutropenia >7 days | Steroid-responsive |
| Grade 3 or 4 immune-related thrombocytopenia, hemolytic anemia, or neutropenia >7 days | None |
| Any Grade 5 | None |
| Other DLT Criteria | |
| Grade 2 immune-related adverse event | Steroid-responsive or adequately controlled with hormone replacement |
| Grade 3 or higher immune-mediated adverse event | Grade 3 hypothyroidism with resolution or adequate control with physiologic hormone replacement or other medical management |
| TTS characterized by both Grade 3 or 4 thrombosis and thrombocytopenia | None |
| Grade 2 or higher Guillain-Barré syndrome | None |

Abbreviations: ALT=alanine aminotransferase; AST=aspartate aminotransferase; DLT=dose-limiting toxicity; NCI-CTCAE=National Cancer Institute Common Terminology Criteria for Adverse Events; TLS=tumor lysis syndrome; TTS=Thrombosis with Thrombocytopenia; ULN=upper limit of normal.
a. If associated with a syndrome described in NCI-CTCAE, Version 5.0, the grade of the syndrome should dictate the DLT assessment.
b. Unscheduled laboratory monitoring should be done to document the resolution of the specific toxicity.

### Inclusion Criteria

Each potential participant must satisfy all of the following criteria to be enrolled in the study:

### Age

1. Be ≥18 years of age (or the legal age of consent in the jurisdiction in which the study is taking place) at the time of informed consent.

### Disease Characteristic

2. Have a diagnosis of any of the following conditions defined by the 2016 WHO criteria (Arber 2016) that meets the stated risk criteria:
Essential thrombocythemia that is not very low risk per NCCN 2021 guidelines. OR
Any of the following types of myelofibrosis:
   primary myelofibrosis that is not low risk (ie, 0), defined by the DIPSS (Passamonti 2010) or DIPSS-PLUS models (Gangat 2011). Participants with PMF age ≤70 years may alternatively enroll as long as they do not have low risk (ie, 0-1) disease per MIPSS-70 (Guglielmelli 2018), and those any age are eligible if their disease is not low or very low risk (ie, 0-2) per MIPSS-70+ (Tefferi 2018a, Tefferi 2018b).
   post-essential thrombocythemia myelofibrosis that is not low risk (ie, <11) defined by the Myelofibrosis Secondary to PV and ET-Prognostic Model (MYSEC-PM) (Passamonti 2017).
   prefibrotic myelofibrosis that is not low risk (ie, 0), defined by the DIPSS (Passamonti 2010) or DIPSS-PLUS models (Gangat 2011), as these models have been previously applied to this population (Mudireddy 2017).
OR (at the time of expansion only with SET approval)
Either polycythemia vera that is not low risk per NCCN 2021 guidelines or post-polycythemia vera myelofibrosis that is not low risk (ie, <11) defined by MYSEC-PM (Passamonti 2017).

3. Be positive for Type 1 or Type 2 CALR mutation or positive for the V617F JAK2 mutation and HLA-A0201 per medical history or local testing. Type 1 mutation involves a 52-base pair deletion (p.L367fs*46), and Type 2 mutation involves a 5-base pair TTGTC insertion (p.K385fs*47).

4. Have an Eastern Cooperative Oncology Group (ECOG) performance status grade of 0 or 1 or 2 (Oken 1982) (Grade 0: Fully active, able to carry on all pre-disease performance without restriction; Grade 1: Restricted in physically strenuous activity but ambulatory and able to carry out work of a light or sedentary nature, e.g., light housework, office work; Grade 2: Ambulatory and capable of all self-care, confined to bed or chair more than 50% of waking hours).

5. Participants should have the following clinical hematology laboratory values predose:
Leukocytes ≥1.5 × 10⁹/L
Neutrophils ≥1.0 × 10⁹/L
Platelets ≥20 × 10⁹/L (minimum of 3 days post transfusion)
Hemoglobin >7 g/dL

6. Participants should have the following clinical chemistry laboratory values predose:
alanine aminotransferase (ALT): ≤3 × ULN
aspartate aminotransferase (AST): ≤3 × ULN
total bilirubin: ≤1.5 × ULN (except participants with Gilbert Syndrome who must have a Total bilirubin level of ≤3.0 × ULN)
renal function: Estimated or measured glomerular filtration rate ≥40 mL/min per MDRD formula

### Sex and Contraceptive/Barrier Requirements

7. A female participant of childbearing potential must have a negative highly sensitive serum β-human chorionic gonadotropin at screening and within 48 hours prior to the first dose of study treatment and must agree to further serum or urine pregnancy tests during the study.

8. A female participant of childbearing potential must agree to all the following during the study and for 6 months after the last dose of study treatment: Use a barrier method of contraception; Use a highly effective preferably user-independent method of contraception; Not to donate eggs (ova, oocytes) or freeze for future use for the purposes of assisted reproduction; Not plan to become pregnant; Not to breast-feed.

9. A male participant must agree to all the following during the study and for 90 days after the last dose of study treatment: Wear a condom when engaging in any activity that allows for passage of ejaculate to another person; Not to father a child; Not to donate sperm or freeze for future use for the purpose of reproduction.

### Exclusion Criteria

Any potential participant who meets any of the following criteria will be excluded from participating in the study:

### Medical Conditions

1. History of any significant medical condition per investigators judgment (eg, severe asthma/COPD, poorly regulated heart condition, insulin dependent diabetes mellitus).

2. Concurrent or recently diagnosed or treated malignancies present at the time of participant screening. Exceptions are squamous and basal cell carcinoma of the skin, carcinoma in situ of the cervix and any malignancy that is considered cured or has minimal risk of recurrence within 1 year of first dose of study drug. Participants cured of another malignant disease with no sign of relapse ≥3 years after treatment ended are allowed to enter the protocol.

3. Any active autoimmune diseases eg, autoimmune neutropenia, inflammatory bowel disease, thrombocytopenia or hemolytic anemia, systemic lupus erythematosus, scleroderma, myasthenia gravis, autoimmune glomerulonephritis, autoimmune neuropathies, rheumatoid arthritis, etc. Enrollment is permitted in the following situations: Vitiligo and adequately controlled endocrine deficiencies such hypothyroidism.

4. Serious known clinically relevant allergies or earlier anaphylactic reactions.

6. Currently pregnant or breastfeeding.

### Prior/Concomitant Therapy

7. Prior treatment with any JAK2 inhibitor.

8. Prior treatment with any checkpoint inhibitor.

9. Known sensitivity or allergies to the heme vaccine (GAd20-CALR-JAK2 and/or MVA-CALR-JAK2) or any of its components.

10. Known sensitivity or contraindications to the use of ipilimumab per local prescribing information.

11. Taking immune suppressive medications including systemic corticosteroids or methotrexate at the time of enrollment.

12. Treatment with chemotherapy or immune therapy (excluding hydroxyurea or anagrelide with stable dose within 3 months prior to screening) and/or presence of toxicities (except for alopecia, peripheral neuropathy, thrombocytopenia, neutropenia, anemia) from previous anticancer therapies that have not resolved to baseline or to Grade 1 or less.

13. Prior treatment with interferon alpha (including PEGylated-IFN-α) within 3 months prior to enrollment.

14. Taken any disallowed therapies (any form of IFN-α (including PEGylated-IFN-α), any JAK2 inhibitor, any checkpoint inhibitor (other than ipilimumab per protocol)), Concomitant Therapy before the planned first dose of study treatment.

15. a. Received or plans to receive any live, attenuated vaccine within 4 weeks before the first dose of study drug or within 2 weeks after the last dose of study drug.

b. Non-live vaccines (eg, influenza) are permitted as late as 2 weeks before the study treatment. Additional non-live vaccines may be administered irrespective of study drug administration following notification. Non-live vaccines approved or authorized for emergency use (eg, SARS-CoV-2 [COVID-19]) by local health authorities are permitted at any point with respect to study treatment.

### Prior/Concurrent Clinical Study Experience

16. Received an investigational treatment or used an invasive investigational medical device within 2 weeks before the planned first dose of study treatment or received an investigational biological product within 3 months or 5 half-lives, whichever is longer, before the planned study treatment, or is currently enrolled in an investigational study.

### Diagnostic Assessments

17. History or evidence of serious active viral, bacterial, or uncontrolled systemic fungal infection requiring parenteral treatment within 7 days before the first dose of study drug or history of chronic bacterial, fungal, or parasitic infection (eg, tuberculosis, candidiasis, helminths).

18. Participant is known to be positive for human immunodeficiency virus (HIV).

19. Active hepatitis B virus (HBV) or hepatitis C virus (HCV) infection according to local laboratory range, on all available tests for the past 6 months or other clinically active liver disease:
Seropositive for HBV: defined by a positive test for hepatitis B surface antigen [HBsAg]. Participants with resolved infection (ie, participants who are HBsAg negative with antibodies to total hepatitis B core antigen [anti-HBc] with or without the presence of hepatitis B surface antibody [anti-HBs]) must be screened using real-time polymerase chain reaction (RT-PCR) measurement of HBV DNA levels. Those who are RT-PCR positive will be excluded. Participants with serologic findings suggestive of HBV vaccination (anti-HBs positivity as the only serologic marker) AND a known history of prior HBV vaccination, do not need to be tested for HBV DNA by RT-PCR.
Known HCV infection or positive serologic testing for HCV (anti-HCV antibody).
Positive HCV antibody test result at screening or within 3 months prior to starting study treatment. NOTE: Participants with positive HCV antibody due to prior resolved disease can be enrolled only if a confirmatory negative HCV RNA test is obtained.
Positive HCV RNA test result at screening or within 3 months prior to first dose of study treatment. NOTE: Test is optional and participants with negative HCV antibody test are not required to also undergo HCV RNA testing.

20. Any condition for which, participation would not be in the best interest of the participant (eg, compromise the well-being) or that could prevent, limit, or confound the protocol-specified assessments.

### Description of Treatments

A description of the study treatments is provided below.

TABLE 6

Description of Treatments

| | | | |
|---|---|---|---|
| Treatment Name | GAd20-CALR-JAK2 | MVA-CALR-JAK2 | ipilimumab |
| Type | Biologic | Biologic | Biologic |
| Dose Formulation | Liquid in vial | Liquid in vial | Liquid in vial |
| Unit Dose Strength(s) | 1×10¹¹ virus particles (vp) | 1.0×10⁸ infectious units (IFU) | 200 mg in a single dose vial |
| Dosage Level(s) | 1×10¹¹ virus particles (VP) | 1×10⁸ infectious units (IFU) | 1 mg/kg 3 mg/kg^{a} |
| Route of Administration | IM injection | IM injection | IV infusion |
| Use | Experimental | Experimental | Experimental |
| IMP | Yes | Yes | Yes |
| NIMP/AxMP | No | No | No |
| Sourcing | Provided centrally by the sponsor | Provided centrally by the sponsor | Provided centrally by the sponsor |
| Packaging and Labeling (Labels will contain information to meet the applicable regulatory requirements.) | Individual Participant Kits | Individual Participant Kits | Individual Participant Kits |
| Delivery Instructions | NA | NA | NA |
| Food/Fasting Requirement | None | None | None |
| Current/Former Name(s) or Alias(es) | GAd20-CALR-JAK2 GAd20-HCal-JAK2 GAd20 HCalJ9.9 | MVA-CALR-JAK2 MVA-mutCALR-mutJAK2 MVA-HCalJ-9.9 | YERVOY^{®} |

^{a} Only to be tested if ipilimumab at 1 mg/kg is tolerated (see Section Error! Reference source not found, and Section 0).

The ipilimumab IV infusion is to be administered first. The IM injection of either vaccine prime or boost is to be administered within 30 minutes to 4 hours after the completion of the ipilimumab infusion. If a participant will not be receiving either the vaccine prime (Gad20-CALR-JAK2) or boost (MVA-CALR-JAK2) at a study visit, they should not receive the administration of ipilimumab.

### Dose Modification

No dose reductions of Gad20-CALR-JAK2 and MVA-CALR-JAK2 are permitted.

The study will be initiated with the priming administration of Gad20-CALR-JAK2 administered in combination with ipilimumab at the 1 mg/kg dose level. In the event of any weight change in excess of an absolute change (+ or -) of 10% from baseline (Week 0 Day 1) or the last dosing weight, the dose of ipilimumab should be recalculated. Weight can be measured up to 48 hours before infusion. The ipilimumab dosing schedule may be adjusted to expand a dosing cohort to further evaluate safety, immunogenicity, efficacy, PK and pharmacodynamic findings at the given dose level. Additional dose levels for ipilimumab (eg, 3 mg/kg) may also be evaluated pending review of emerging safety and efficacy data including antigen-specific T-cell response.

Toxicities attributed to ipilimumab should be managed by permanently discontinuing the ipilimumab; however, dose reductions of ipilimumab from 3 mg/kg to 1 mg/kg may be reviewed and approved on a case-by-case basis. The only dosing options for ipilimumab are none (0 mg/kg), 1.0 mg/kg, or 3 mg/kg.

### Permitted Therapies

The following are examples of supportive therapies that may be used during the study:
Hydroxyurea may be used at screening and during the first 2 Treatment Cycles as needed to reduce WBC counts to ≤20 × 10⁹/L.
Standard supportive care therapies for prophylaxis or as treatment (ie, antiemetics, antidiarrheals, anticholinergics, antispasmodics, antipyretics, antihistamines, analgesics, antibiotics, antifungals, and other antimicrobials, and other medications intended to treat symptoms or signs of disease or AEs) as clinically indicated, according to institutional standards and as deemed necessary.
Anti-thrombotic medications such as acetylsalicylic acid or anticoagulants such as enoxaparin or coumadin.
Documented infectious complications should be treated with oral or IV antibiotics or other anti-infective agents as considered appropriate by the treating investigator for a given infectious condition, according to standard institutional practice.
Growth factor support (including granulocyte colony stimulating factor [G-CSF]), erythropoietin-stimulating agents, and transfusions such as RBCs and platelets are permitted as prophylaxis or to treat symptoms or signs of neutropenia, anemia, or thrombocytopenia.
Proton pump inhibitors, histamine receptor (H₂) antagonists, and antacids.
Therapeutic phlebotomy.

### Prohibited or Restricted Therapies

The following medications are prohibited or restricted during the study:
Any form of IFN-α (including PEGylated-IFN-α)
Any JAK2 inhibitor
Any checkpoint inhibitor (other than ipilimumab per protocol)

### Efficacy and Immunogenicity Assessments

### Efficacy Assessments

Assessment of disease includes the evaluations described below. Efficacy evaluations will include physical examination, overall clinical response per revised response criteria by the International Working Group-Myeloproliferative Neoplasms Research and Treatment (IWG-MRT) and ELN consensus report, Total Symptom Score, disease burden as defined by IWG and ELN response criteria at Week 24 and Week 48, peripheral blood mutCALR and JAK2V617F burden, bone marrow response, clinical symptoms, and time to progression or time to initiation of next therapy. In addition, the antigen-specific immune responses to the mutCALR and JAK2V617F mutations will be evaluated.

### Bone Marrow Assessment

A bone marrow sample is required at screening and at various timepoints. Bone marrow aspirate and biopsy are preferred at all disease evaluations.

Screening bone marrow results will need to include blast burden, cell differential, fibrosis grading (refer to Arber 2016), cytogenetics, and molecular testing. For subsequent disease assessments, the local results will need to include blast burden, cell differential, and fibrosis grading. The bone marrow assessment at the EOT visit may be omitted if the participant previously underwent a disease assessment within the prior 8 weeks.

Collectively, bone marrow assessments, liver, and spleen assessments, total symptom score, peripheral blood results, and thrombotic events will be used to assign a disease response per applicable IWG-MRT criteria.

### Liver and Spleen Assessments

At screening and during all disease evaluation visits, a spleen and liver assessment by physical exam will be required and will include measurements of organomegaly below the costal margin.

At screening and all disease evaluation visits, an abdominal ultrasound that assesses the liver and spleen will be required. CT or MRI are permitted, but ultrasound is the preferred. These ultrasounds will document the presence or absence of hepatomegaly. In addition, measurements of the spleen (both the longest dimension and either an estimated volume or listing of width, thickness, and craniocaudal length) will be included. Spleen volume estimates will be derived as previously reported (Yetter 2003).

### Total Symptom Score

Symptom burden will be assessed regularly by the 7-day recall Myelofibrosis Symptom Assessment Form version 4.0. This paper-based assessment will occur at screening, every week (±1 day) from enrollment (Week 0) through Week 24, every 4 weeks (±1 day) Week 28 through the End of Treatment, and at each long-term follow-up visit.

### Assessment of Disease Response and Progressive Disease

Disease will be evaluated according to the revised response criteria by the International Working Group-Myeloproliferative Neoplasms Research and Treatment (IWG-MRT) and European LeukemiaNet (ELN) consensus report, disease burden at Week 24 and Week 48, peripheral blood mutCALR and JAK2V617F burden, bone marrow response, clinical symptoms, and time to progression or time to initiation of next therapy.

### Biomarkers

The magnitude and type of adaptive T-cell immune response to the antigens included in the vaccine will be evaluated using immune assays such as IFN-g ELISpot and intracellular cytokine staining (ICS). In addition, activation induced marker (AIM) assay or TCR sequencing may be performed to measure antigen-specific immune responses. Serum samples will be collected to analyze, but not limited to, changes in cytokine levels and antigen-specific antibodies to further understand the activity of the heme vaccine.

Genomic DNA will be prepared from peripheral blood to characterize the mutation status on a panel of myeloid-related genes at screening and post-treatment by next-generation sequencing (NGS) or other methodology where necessary to evaluate the association with clinical response. Mutant Allele Burden (MAB) for mutant CALR, and V617F JAK2 mutation will be evaluated to understand the clinical activity of the heme vaccine. The mutation status of genes including but not limited to ASXL1, DNMT3A, EZH2, IDH1, IDH2, MPL, RUNX1, SF3Bl, SH2B3, SRSF2, TET2, TP53, and U2AF1 may be assessed. Adjustments regarding the genes analyzed may be made based on evidence from emerging data. In addition, HLA typing may be performed to assess the correlation with treatment response.

Additional biomarkers may be evaluated in bone marrow, whole blood, plasma, serum and RNA or protein to further understand treatment responses.

### Immunogenicity Assessments

The immune response to the components of the heme vaccine regimen (e.g., anti-GAd20/anti-hexon anti-vector antibodies) will be evaluated using immune assays such as IFN-g ELISpot, ELISA.

Venous blood samples for the measurement of serum concentrations of anti-GAd20 neutralizing antibodies (and possibly for anti-MVA neutralizing antibodies) will be collected at various time points. The detection and characterization of anti-GAd20 neutralizing antibodies (and potentially anti-MVA neutralizing antibodies) will be performed using a validated assay method.

### Risk Stratification Approaches Based on Underlying Disease

### Myelofibrosis

For participants with an underlying diagnosis of myelofibrosis:

TABLE 7

Dynamic International Prognostic Scoring System (DIPSS)
DIPSS for survival in primary myelofibrosis
Value

| | | | |
|---|---|---|---|
| Prognostic variable | 0 | 1 | 2 |
| Age (years) | ≤65 | >65 | |
| White blood cell count, ×10⁹/L | ≤25 | >25 | |
| Hemoglobin, g/dL | ≥10 | | <10 |
| Peripheral blood blast, % | <1 | ≥1 | |
| Constitutional symptoms, Y/N | N | Y | |

The risk category is obtained adding up the values of each prognostic variable. Risk categories are defined as low: 0, intermediate-1: 1 or 2, intermediate -2: 3 or 4, and high: 5 or 6.
Adapted from: Passamonti 2010.
Online calculator for DIPSS: qxmd.com/calculate/calculator_187/dipss-prognosis-in-myelofibrosis.

TABLE 8

Dynamic International Prognostic Scoring System-Plus (DIPSS-PLUS)
DIPSS-PLUS for primary myelofibrosis

| Prognostic variable | Points |
|---|---|
| DIPSS low-risk | 0 |
| DIPSS intermediate-risk 1 | 1 |
| DIPSS intermediate-risk 2 | 2 |
| DIPSS high-risk | 3 |
| Platelets < 100 × 10⁹/L | 1 |
| Transfusion need | 1 |
| Unfavorable karyotype* | 1 |

The risk category is obtained adding up the values of each prognostic variable. Risk categories are defined as low: 0, intermediate-1: 1, intermediate -2: 2 or 3, and high: 4 to 6.
*Unfavorable karyotype: complex karyotype or sole or two abnormalities that include trisomy 8, 7/7q-, i(17q), 5/5q-, 12p-, inv(3), or 11q23 rearrangement.
Adapted from: Gangat 2011.
Online calculator for DIPSS-PLUS: qxmd.com/calculate/calculator_315/dipss-plus-score-for-prognosis-in-myelofibrosis

TABLE 9

Mutation-Enhanced IPSS (MIPSS-70) for Patients with PMF Age ≤70 Years
MIPSS-70

| Prognostic variable | Points |
|---|---|
| Hemoglobin <10 g/dL | 1 |
| Leukocytes >25 × 10⁹/L | 2 |
| Platelets < 100 × 10⁹/L | 2 |
| Circulating blasts ≥2% | 1 |
| Bone marrow fibrosis grade ≥2 | 1 |
| Constitutional symptoms | 1 |
| CALR type-1 unmutated genotype | 1 |
| High-molecular risk (HMR) mutations⁺ | 1 |
| ≥2 HMR mutations | 2 |

The risk category is obtained adding up the values of each prognostic variable. Risk categories are defined as low: 0-1, intermediate: 2-4, and high: ≥5.
⁺Presence of a mutation in any of the following genes: ASXL1, EZH2, SRSF2, or IDH½. Adapted from: Guglielmelli 2018; Online calculator for MIPSS-70: mipss70score.it/

TABLE 10

Mutation and Karyotype-Enhanced IPSS (MIPSS-70+ Version 2.0) for Patients with PMF
MIPSS-70+ Version 2.0

| Prognostic variable | Points |
|---|---|
| Severe anemia (Hemoglobin <8 g/dL in women and <9 g/dL in men) | 2 |
| Moderate anemia (Hemoglobin 8-9.9 g/dL in women and 9-10.9 g/dL in men) | 1 |
| Circulating blasts ≥2% | 1 |
| Constitutional symptoms | 2 |
| Absence of CALR-1 type mutation | 2 |
| HMR mutations⁺ | 2 |
| ≥2 HMR mutations | 3 |
| Unfavorable karyotype* | 3 |
| Very-high-risk (VHR) karyotype⁺⁺ | 4 |

The risk category is obtained adding up the values of each prognostic variable. Risk categories are defined as very low: 0, low: 1-2, intermediate: 3-4, high: 5-8, and very high: ≥9.
+Presence of a mutation in any of the following genes: ASXL1, EZH2, SRSF2, or IDH½.
Unfavorable karyotype: complex karyotype or sole or two abnormalities that include trisomy 8, 7/7q-, i(17q), 5/5q-, 12p-, inv(3), or 11q23 rearrangement.
⁺⁺VHR karyotype: single/multiple abnormalities of -7, i(17q), inv(3)/3q21, 12p-/12p11.2, 11q-/11q23, or other autosomal trisomies not including +8/+9 (eg, +21, +19).
Adapted from: Tefferi 2018a and Tefferi 2018b.
Online calculator for MIPSS-70+ Version 2.0: mipss70score.it/

TABLE 11

Risk Stratification for Patients with Post-PV and Post-ET MF
Myelofibrosis secondary to PV and ET-prognostic model (MYSEC-PM)

| Prognostic variable | Points |
|---|---|
| Age at diagnosis | 0.15 per patient’s year of age |
| Hemoglobin <10 g/dL | 2 |
| Circulating blasts ≥3% | 2 |
| Absence of CALR-1 type mutation | 2 |
| Platelets < 150 × 10⁹/L | 1 |
| Constitutional symptoms | 1 |

The risk category is obtained adding up the values of each prognostic variable. Risk categories are defined as low: <11, intermediate-1: ≥11 and <14, intermediate-2: ≥14 and <16, and high: ≥16.
Adapted from: Passamonti 2017.
Online calculator for MYSEC-PM: mysec-pm.eu/

### Essential Thrombocythemia

For participants with an underlying diagnosis of essential thrombocythemia:

TABLE 12

Revised International Prognostic Score of Thrombosis for ET (IPSET-thrombosis)

| | |
|---|---|
| Very-low-risk | Age ≤60 years, no JAK2 mutation, no prior history of thrombosis |
| Low-risk | Age ≤60 years, with JAK2 mutation, no prior history of thrombosis |
| Intermediate-risk | Age >60 years, no JAK2 mutation, no prior history of thrombosis |
| High-risk | History of thrombosis at any age or age >60 years with JAK2 mutation |

Adapted from NCCN 2021 guidelines.

### Polycythemia Vera

For participants with an underlying diagnosis of polycythemia vera:

TABLE 13

Vascular and Neoplastic Risk for Patients with Polycythemia Vera

| | |
|---|---|
| Low-risk | age <60 years and no prior history of thrombosis |
| High-risk | age ≥60 years and/or prior history of thrombosis |

Adapted from NCCN 2021 guidelines.

### Response Criteria for Essential Thrombocythemia (ET), Polycythemia Vera (PV), and Myelofibrosis (MF)

TABLE 14

Response criteria for Essential Thrombocythemia (ET)
Criteria
Complete remission

| | |
|---|---|
| A | Durable (≥12 weeks) resolution of disease-related signs including palpable hepatosplenomegaly, large symptoms improvement (≥7-point decrease in MFSAF v 4.0 [Gwaltney 2017]), AND |
| B | Durable (≥12 weeks) peripheral blood count remission, defined as platelet count ≤400 × 10⁹/L, WBC count <10 × 10⁹/L, absence of leukoerythroblastosis, AND |
| C | Without signs of progressive disease, and absence of any hemorrhagic or thrombotic events, AND |
| D | Bone marrow histological remission defined as disappearance of megakaryocyte hyperplasia and absence of >Grade 1 reticulin fibrosis. |

Partial remission
All signs A-C from CR (above) AND
Without bone marrow histological remission, defined as the persistence of megakaryocyte hyperplasia.

| | |
|---|---|
| No response | Any response that does not satisfy partial remission |
| Progressive disease | Transformation into PV (Arber 2016), post-ET myelofibrosis (Barosi 2008), myelodysplastic syndrome (Arber 2016), or acute leukemia (Arber 2016) |

Molecular response is not required for assignment as complete remission or partial remission. Molecular response evaluation requires analysis in peripheral blood granulocytes. Complete molecular response is defined as eradication of a pre-existing abnormality. Partial molecular response applies only to patients with at least 20% mutant allele burden at baseline. Partial response is defined as ≥50% decrease in allele burden. WBC=white blood cell
NOTE: The results obtained for the disease evaluation visit will be used to assign a timepoint response for that visit that does not include durability, while the overall response will include durability of response.
Table adapted from Barosi 2013.

TABLE 15

Response criteria for Polycythemia Vera (PV)

| | Criteria |
|---|---|
| Complete remission | |
| A | Durable (≥12 weeks) resolution of disease-related signs including palpable hepatosplenomegaly, large symptoms improvement (≥7-point decrease in MFSAF v 4.0 [Gwaltney 2017]), AND |
| B | Durable (≥12 weeks) peripheral blood count remission, defined as hematocrit lower than 45% without phlebotomies, platelet count ≤400 × 10⁹/L, WBC count <10 × 10⁹/L, AND |
| C | Without signs of progressive disease, and absence of any hemorrhagic or thrombotic events, AND |
| D | Bone marrow histological remission defined as the presence of age-adjusted normocellularity and disappearance of trilinear hyperplasia, and absence of >Grade 1 reticulin fibrosis. |

Partial remission
All signs A-C from CR (above) AND
Without bone marrow histological remission, defined as the persistence of megakaryocyte hyperplasia.

| | |
|---|---|
| No response | Any response that does not satisfy partial remission |
| Progressive disease | Transformation into post-PV myelofibrosis (Barosi 2008), myelodysplastic syndrome (Arber 2016), or acute leukemia (Arber 2016) |

Molecular response is not required for assignment as complete remission or partial remission. Molecular response evaluation requires analysis in peripheral blood granulocytes. Complete molecular response is defined as eradication of a pre-existing abnormality. Partial molecular response applies only to patients with at least 20% mutant allele burden at baseline. Partial response is defined as ≥50% decrease in allele burden. WBC=white blood cell
NOTE: The results obtained for the disease evaluation visit will be used to assign a timepoint response for that visit that does not include durability, while the overall response will include durability of response.
Table adapted from Barosi 2013.

TABLE 16

Revised IWG-MRT and ELN Response Criteria for Myelofibrosis (MF)

| | |
|---|---|
| Response categories | Required criteria (for all response categories, benefit must last ≥12 weeks to qualify) |
| Complete Remission | Bone marrow:* Age-adjusted normocellularity, <5% blasts, ≤Grade 1 MF, resolution of leukoerythroblastosis, AND Peripheral blood: Hemoglobin ≥10 g/dL and <ULN, neutrophil count ≥ 1 × 10⁹/L and <ULN, Platelet count ≥100 × 10⁹/L and <ULN, <2%immature myeloid cells‡, AND Clinical: Resolution of disease symptoms, spleen and liver not palpable, no evidence of EMH |
| Partial Remission | Peripheral blood: Hemoglobin ≥10 g/dL and <ULN, neutrophil count ≥1 × 10⁹/L and <ULN, platelet count ≥100 × 10⁹/L and <ULN, <2% immature myeloid cells‡, AND Clinical: Resolution of disease symptoms, spleen and liver not palpable, no evidence of EMH, OR Bone marrow: Age-adjusted normocellularity, <5% blasts, ≤Grade 1 MF, AND Peripheral blood: Hemoglobin ≥8.5 but <10 mg/L and <ULN, neutrophil count ≥1 × 10⁹/L and <ULN, platelet count ≥50, but <100 × 10⁹/L and <ULN, <2% immature myeloid cells‡, AND Clinical: Resolution of disease symptoms, spleen and liver not palpable, no evidence of EMH |
| Clinical improvement | The achievement of anemia, spleen or symptoms response without progressive disease or increase in severity of anemia, thrombocytopenia, or neutropenia§ |
| Anemia response | Transfusion-independent patients: ≥2 g/dL increase in hemoglobin level∥ Transfusion-dependent patients: becoming transfusion-independent (refer to Section Error! Reference source not found.). |
| Spleen response# | A baseline splenomegaly that is palpable at 5-10 cm, below the LCM, becomes not palpable** OR A baseline splenomegaly that is palpable at >10 cm, below the LCM, decreases by ≥50%** A baseline splenomegaly that is palpable at <5 cm, below the LCM, is not eligible for spleen response A spleen response requires confirmation by ultrasound or other imaging modality showing ≥35% spleen volume reduction |
| Symptoms response | A ≥50% reduction in the MFSAF v4.0 (Gwaltney 2017)†† |
| Progressive disease‡‡ | Appearance of a new splenomegaly that is palpable at least 5 cm below the LCM OR A ≥100% increase in palpable distance, below LCM, for baseline splenomegaly of 5-10 cm OR A 50% increase in palpable distance, below LCM, for baseline splenomegaly of >10 cm OR Leukemic transformation confirmed by a bone marrow blast count of ≥20% OR A peripheral blood blast content of ≥20% associated with an absolute blast count of ≥1 × 10(9)/L that lasts for ≥2 weeks |
| Stable disease | Belonging to none of the above listed response categories |
| Relapse | No longer meeting criteria for at least CI after achieving CR, PR, or CI, OR Loss of anemia response persisting for at least 1 month OR Loss of spleen response persisting for at least 1 month |

Recommendations for assessing treatment-induced cytogenetic and molecular changes

| | |
|---|---|
| Cytogenetic remission | At least 10 metaphases must be analyzed for cytogenetic response evaluation and requires confirmation by repeat testing within 6 months window CR: eradication of a pre-existing abnormality PR: ≥50% reduction in abnormal metaphases (partial response applies only to patients with at least 10 abnormal metaphases at baseline) |
| Molecular remission | Molecular response evaluation must be analyzed in peripheral blood granulocytes and requires confirmation by repeat testing within 6 months window CR: Eradication of a pre-existing abnormality PR: ≥50% decrease in allele burden (partial response applies only to patients with at least 20% mutant allele burden at baseline) |
| Cytogenetic/molecular relapse | Re-emergence of a pre-existing cytogenetic or molecular abnormality that is confirmed by repeat testing |

Table adapted from Tefferi 2016.
NOTE: The results obtained for the disease evaluation visit will be used to assign a timepoint response for that visit that does not include durability, while the overall response will include durability of response.
CI, Clinical Improvement; CR, Clinical Remission; EMH, extramedullary hematopoiesis (no evidence of EMH implies the absence of pathology- or imaging study-proven nonhepatosplenic EMH); LCM, left costal margin; MF, Myelofibrosis; PR, Partial Remission; ULN, upper limit of normal.
*Cytogenetic and molecular responses are not required for CR assignment.
‡Immature myeloid cells constitute blasts + promyelocytes + myelocytes + metamyelocytes + nucleated red blood cells. In splenectomized patients, <5% immature myeloid cells is allowed.
§Increase in severity of anemia constitutes the occurrence of new transfusion dependency or a ≥2 g/dL decrease in hemoglobin level from pretreatment baseline that lasts for ≥12 weeks. Increase in severity of thrombocytopenia or neutropenia is defined as a 2-grade decline, from pretreatment baseline, in platelet count or absolute neutrophil count, according to the CTCAE version 5.0. In addition, assignment to CI requires a minimum platelet count of ≥25,000 × 10(9)/L and absolute neutrophil count of ≥0.5 × 10(9)/L.
∥Applicable only to patients with baseline hemoglobin of <10 g/dL. In patients not meeting the strict criteria for transfusion dependency at the time of study enrollment, but have received transfusions within the previous month, the pretransfusion hemoglobin level should be used as the baseline.
#In splenectomized patients, palpable hepatomegaly is substituted with the same measurement strategy.
**Spleen or liver responses must be confirmed by imaging studies where a ≥35% reduction in spleen volume, as assessed by ultrasound (or other imaging modality), is required. Furthermore, a ≥35% volume reduction in the spleen or liver, by ultrasound or other imaging modality, constitutes a response regardless of what is reported with physical examination.
‡‡Progressive disease assignment for splenomegaly requires confirmation by ultrasound or other imaging modality showing a ≥25% increase in spleen volume from baseline.
Baseline values for both physical examination and imaging studies refer to pretreatment baseline and not to post-treatment measurements.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 17

Sequences

| | |
|---|---|
| SEQ ID NO: | Sequence |
| 1 CALR-JAK2 transgene | MKDKQDEEQRTRRMMRTKMRMRRMRRTRRKMRRKMSPARPRTSCREACLQGWTEAAYEEAEDNCRRMMRTKAAYVLNYGVCFCAAYFCGDENILV |
| 2 CALR-JAK2 transgene | ATGAAGGACAAACAGGACGAGGAACAGAGAACAAGGCGGATGATGAGAACCAAGATGAGGATGAGACGGATGCGCAGAACACGGAGGAAAATGAGAAGGAAGATGTCTCCTGCCAGACCTAGGACCAGCTGTAGAGAAGCTTGTCTGCAGGGATGGACAGAAGCCGCTTACGAAGAGGCCGAAGACAACTGTCGGAGAATGATGCGGACAAAAGCTGCCTACGTGCTGAATTATGGCGTGTGTTTTTGCGCCGCATACTTTTGTGGCGACGAGAACATCCTGGTG |
| 3 CALR epitope 1 | MKDKQDEEQRTRRMMRTKMRMRRMRRTRRKMRRKMSPARPRTSCREACLQGWTE |
| 4 CALR epitope 2 | EEAEDNCRRMMRTK |
| 5 JAK2 epitope 1 | VLNYGVCFC |
| 6 JAK 2 epitope 2 | FCGDENILV |
| 7 AAY linker | AAY |
| 8 CALR-JAK2 transgene with TCE (“GAd20-HCalJ-9.9”) | MACPGFLWALVISTCLEFSMAMKDKQDEEQRTRRMMRTKMRMRRMRRTRRKMRRKMSPARPRTSCREACLQGWTEAAYEEAEDNCRRMMRTKAAYVLNYGVCFCAAYFCGDENILV |
| 9 CALR-JAK2 transgene with TCE (“GAd20-HCalJ-9.9”) | ATGGCTTGTCCTGGCTTTCTGTGGGCCCTGGTGATCAGCACATGTCTGGAGTTCTCTATGGCTATGAAGGACAAACAGGACGAGGAACAGAGAACAAGGCGGATGATGAGAACCAAGATGAGGATGAGACGGATGCGCAGAACACGGAGGAAAATGAGAAGGAAGATGTCTCCTGCCAGACCTAGGACCAGCTGTAGAGAAGCTTGTCTGCAGGGATGGACAGAAGCCGCTTACGAAGAGGCCGAAGACAACTGTCGGAGAATGATGCGGACAAAAGCTGCCTACGTGCTGAATTATGGCGTGTGTTTTTGCGCCGCATACTTTTGTGGCGACGAGAACATCCTGGTG |
| 10 TCE | MACPGFLWALVISTCLEFSMA |
| 11 TCE | ATGGCTTGTCCTGGCTTTCTGTGGGCCCTGGTGATCAGCACATGTCTGGAGTTCTCTATGGCT |
| 12 CALR-JAK2 transgene with TCE (“MVA-HCalJ-9.9”) | MGQKEQIHTLQKNSERMSKQLTRSSQAVMKDKQDEEQRTRRMMRTKMRMRRMRRTRRKMRRKMSPARPRTSCREACLQGWTEAAYEEAEDNCRRMMRTKAAYVLNYGVCFCAAYFCGDENILV |
| 13 CALR-JAK2 transgene with TCE (“MVA-HCalJ-9.9”) | ATGGGCCAGAAGGAACAGATTCATACGCTTCAGAAAAATTCTGAACGAATGTCAAAGCAATTGACACGAAGTTCTCAGGCAGTAATGAAGGACAAACAAGACGAAGAACAACGAACTAGGCGGATGATGAGGACTAAGATGAGGATGCGGAGGATGAGACGGACGCGACGCAAGATGCGCCGGAAAATGTCTCCCGCCCGGCCAAGGACGTCTTGTCGGGAAGCCTGTTTGCAGGGCTGGACCGAAGCAGCTTACGAAGAAGCAGAAGACAATTGTCGGCGAATGATGAGAACGAAGGCTGCTTACGTGCTTAACTATGGAGTGTGCTTCTGCGCTGCCTATTTCTGCGGAGATGAGAACATTCTGGTG |
| 14 TCE | MGQKEQIHTLQKNSERMSKQLTRSSQAV |
| 15 TCE | ATGGGCCAGAAGGAACAGATTCATACGCTTCAGAAAAATTCTGAACGAATGTCAAAGCAATTGACACGAAGTTCTCAGGCAGTA |

### EMBODIMENTS

The following list of embodiments is intended to complement, rather than displace or supersede, the previous descriptions.

Embodiment 1. A method of treating or preventing a myeloproliferative disease, a cancer, or a cardiovascular disease, or inducing an immune response, in a subject having a JAK2V617F substitution and/or a CALR exon 9 mutation, the method comprising administering to the subject a treatment regimen comprising:
two or more vaccines comprising a great ape adenovirus serotype 20 (GAd20) virus that, in turn, comprises a nucleotide sequence encoding the amino acid sequence of SEQ ID NO: 1 and
one or more vaccines comprising a Modified Vaccinia Ankara (MVA) virus that, in turn, comprises a nucleotide sequence encoding the amino acid sequence of SEQ ID NO: 1 to thereby treat or prevent the myeloproliferative disease, cancer, or cardiovascular disease, or induce the immune response.

Embodiment 2. The method of embodiment 1, further comprising administering the treatment regimen two or more times.

Embodiment 3. The method of embodiment 1 or 2, further comprising administering:
one or more vaccines comprising the GAd20 virus,
one or more vaccines comprising the MVA virus, or
one or more vaccines comprising the GAd20 virus and one or more vaccines comprising the MVA virus.

Embodiment 4. The method of embodiment 3, comprising administering two vaccines comprising the GAd20 virus and one vaccine comprising the MVA virus.

Embodiment 5. The method of embodiment 3, comprising administering one vaccine comprising the GAd20 virus and one vaccine comprising the MVA virus.

Embodiment 6. The method of embodiment 3, comprising administering three vaccines comprising the MVA virus.

Embodiment 7. The method of embodiment 3, comprising administering two vaccines comprising the MVA virus.

Embodiment 8. The method of any one of the previous embodiments, further comprising administering one or more vaccines comprising the MVA virus.

Embodiment 9. The method of any one of the previous embodiments, wherein each of the vaccines comprising the GAd20 virus comprises about 1 × 10⁹ viral particles (VP) to about 1 × 10¹³ VP of the GAd20 virus.

Embodiment 10. The method of any one of the previous embodiments, wherein each of the vaccines comprising the MVA virus comprises about 1 × 10⁶ infectious units (IFU) to about 1 × 10¹⁰ IFU of the MVA virus.

Embodiment 11. The method of any one of the previous embodiments, further comprising administering an anti-CTLA4 antibody.

Embodiment 12. The method of embodiment 11, comprising administering the anti-CTLA4 antibody with:
the vaccines comprising the GAd20 virus;
the vaccines comprising the MVA virus; or
both.

Embodiment 13. The method of embodiment 11 or 12, comprising administering 0.5 mg/kg to 5 mg/kg of the anti-CTLA4 antibody.

Embodiment 14. The method of any one of embodiments 1-10, further comprising administering an anti-PD-1 antibody.

Embodiment 15. The method of embodiment 14, comprising administering the anti-PD-1 antibody with:
the vaccines comprising the GAd20 virus;
the vaccines comprising the MVA virus; or
both.

Embodiment 16. The method of embodiment 14 or 15, comprising administering 0.5 mg/kg to 5 mg/kg of the anti-PD-1 antibody.

Embodiment 17. The method of any one of the previous embodiments, wherein each of the one or more GAd20 viruses comprise the nucleotide sequence of SEQ ID NO: 2.

Embodiment 18. The method of embodiment 17, wherein the nucleotide sequence further comprises an N-terminal TCE.

Embodiment 19. The method of any one of the previous embodiments, wherein each of the one or more MVA viruses comprise the nucleotide sequence of SEQ ID NO: 2.

Embodiment 20. The method of embodiment 19, wherein the nucleotide sequence further comprises an N-terminal TCE.

Embodiment 21. The method of any one of the previous embodiments, comprising administering a vaccine comprising the GAd20 virus at week 0 and about week 3 and administering a vaccine comprising the MVA virus at about week 9.

Embodiment 22. The method of embodiment 21, comprising administering a vaccine comprising the GAd20 virus at about week 15 and about week 18 and administering a vaccine comprising the MVA virus at about week 24.

Embodiment 23. The method of embodiment 21, comprising administering a vaccine comprising the GAd20 virus at about week 15 and administering a vaccine comprising the MVA virus at about week 24.

Embodiment 24. The method of embodiment 21, comprising administering a vaccine comprising the MVA virus at about week 15, about week 18, and about week 24.

Embodiment 25. The method of embodiment 21, comprising administering a vaccine comprising the MVA virus at about week 15 and about week 24.

Embodiment 26. The method of any one of the previous embodiments, comprising administering a vaccine comprising the MVA virus at about week 36, about week 48, and about week 60.

Embodiment 27. The method of any one of the previous embodiments, comprising administering a vaccine comprising 1 × 10¹¹ VP of the GAd20 virus at week 0 and about week 3 and administering a vaccine comprising 1 × 10⁸ IFU of the MVA virus at about week 9.

Embodiment 28. The method of embodiment 27, comprising administering a vaccine comprising 1 × 10¹¹ VP of the GAd20 virus at about week 15 and about week 18 and administering a vaccine comprising 1 × 10⁸ IFU of the MVA virus at about week 24.

Embodiment 29. The method of embodiment 27, comprising administering a vaccine comprising 1 × 10¹¹ VP of the GAd20 virus at about week 15 and administering a vaccine comprising 1 × 10⁸ IFU of the MVA virus at about week 24.

Embodiment 30. The method of embodiment 27, comprising administering a vaccine comprising 1 × 10⁸ IFU of the MVA virus at about week 15, about week 18, and about week 24.

Embodiment 31. The method of embodiment 27, comprising administering a vaccine comprising 1 × 10⁸ IFU of the MVA virus at about week 15 and about week 24.

Embodiment 32. The method of any one of the previous embodiments, comprising administering a vaccine comprising 1 × 10⁸ IFU of the MVA virus at about week 36, about week 48, and about week 60.

Embodiment 33. The method of embodiment 32, comprising administering one or more further vaccines comprising 1 × 10⁸ IFU of the MVA virus.

Embodiment 34. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10¹¹ VP of the GAd20 virus at about week 15 and about week 18;
a vaccine comprising 1 × 10⁸ IFU of the MVA virus at about week 24; and
a vaccine comprising 1 × 10⁸ IFU of the MVA virus at about week 36, about week 48, and about week 60.

Embodiment 35. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10¹¹ VP of the GAd20 virus at about week 15;
a vaccine comprising 1 × 10⁸ IFU of the MVA virus at about week 24; and
a vaccine comprising 1 × 10⁸ IFU of the MVA virus at about week 36, about week 48, and about week 60.

Embodiment 36. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10⁸ IFU of the MVA virus at about week 15, about week 18, and about week 24; and
a vaccine comprising 1 × 10⁸ IFU of the MVA virus at about week 36, about week 48, and about week 60.

Embodiment 37. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10⁸ IFU of the MVA virus at about week 15 and about week 24; and
a vaccine comprising 1 × 10⁸ IFU of the MVA virus at about week 36, about week 48, and about week 60.

Embodiment 38. The method of any one of embodiments 34-37, further comprising administering an anti-CTLA4 antibody.

Embodiment 39. The method of embodiment 38, comprising administering the anti-CTLA4 antibody with:
the vaccines comprising the GAd20 virus;
the vaccines comprising the MVA virus; or
both.

Embodiment 40. The method of embodiment 38 or 39, comprising administering 1 mg/kg to 3 mg/kg of the anti-CTLA4 antibody.

Embodiment 41. The method of any one of embodiments 34-37, further comprising administering an anti-PD-1 antibody.

Embodiment 42. The method of embodiment 41, comprising administering the anti-PD-1 antibody with:
the vaccines comprising the GAd20 virus;
the vaccines comprising the MVA virus; or
both.

Embodiment 43. The method of embodiment 41 or 42, comprising administering 1 mg/kg to 3 mg/kg of the anti-PD-1 antibody.

Embodiment 44. The method of any one of embodiments 34 to 43, wherein the GAd20 virus comprises the nucleotide sequence of SEQ ID NO: 2.

Embodiment 45. The method of embodiment 44, wherein the nucleotide sequence further comprises an N-terminal TCE.

Embodiment 46. The method of any one of embodiments 34 to 43, wherein the MVA virus comprises the nucleotide sequence of SEQ ID NO: 2.

Embodiment 47. The method of embodiment 46, wherein the nucleotide sequence further comprises an N-terminal TCE.

Embodiment 48. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10¹¹ VP of the GAd20 virus at about week 15 and about week 18;
1 mg/kg to 3 mg/kg of an anti-CTLA4 antibody and a vaccine comprising 1 × 10⁸ IFU of the MVA virus at about week 24; and
1 mg/kg to 3 mg/kg of an anti-CTLA4 antibody and a vaccine comprising 1 × 10⁸ IFU of the MVA virus at about week 36, about week 48, and about week 60.

Embodiment 49. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10¹¹ VP of the GAd20 virus at about week 15 and about week 18;
1 mg/kg to 3 mg/kg of an anti-PD1 antibody and a vaccine comprising 1 × 10⁸ IFU of the MVA virus at about week 24; and
1 mg/kg to 3 mg/kg of an anti-PD1 antibody and a vaccine comprising 1 × 10⁸ IFU of the MVA virus at about week 36, about week 48, and about week 60.

Embodiment 50. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10¹¹ VP of the GAd20 virus at about week 15;
1 mg/kg to 3 mg/kg of an anti-CTLA4 antibody and a vaccine comprising 1 × 10⁸ IFU of the MVA virus at about week 24; and
1 mg/kg to 3 mg/kg of an anti-CTLA4 antibody and a vaccine comprising 1 × 10⁸ IFU of the MVA virus at about week 36, about week 48, and about week 60.

Embodiment 51. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10¹¹ VP of the GAd20 virus at about week 15;
1 mg/kg to 3 mg/kg of an anti-PD1 antibody and a vaccine comprising 1 × 10⁸ IFU of the MVA virus at about week 24; and
1 mg/kg to 3 mg/kg of an anti-PD1 antibody and a vaccine comprising 1 × 10⁸ IFU of the MVA virus at about week 36, about week 48, and about week 60.

Embodiment 52. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10⁸ IFU of the MVA virus at about week 15, about week 18, and about week 24; and
1 mg/kg to 3 mg/kg of an anti-CTLA4 antibody and a vaccine comprising 1 × 10⁸ IFU of the MVA virus at about week 36, about week 48, and about week 60.

Embodiment 53. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10⁸ IFU of the MVA virus at about week 15, about week 18, and about week 24; and
1 mg/kg to 3 mg/kg of an anti-PD1 antibody and a vaccine comprising 1 × 10⁸ IFU of the MVA virus at about week 36, about week 48, and about week 60.

Embodiment 54. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-CTLA4 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-CTLA4 antibody and a vaccine comprising 1 × 10⁸ IFU of the MVA virus at about week 15 and about week 24; and
1 mg/kg to 3 mg/kg of an anti-CTLA4 antibody and a vaccine comprising 1 × 10⁸ IFU of the MVA virus at about week 36, about week 48, and about week 60.

Embodiment 55. A method of treating or preventing a myeloproliferative disease, a cancer, or a cardiovascular disease, or inducing an immune response, in a subject having a JAK2V617F substitution and/or a CALR exon 9 mutation, the method comprising administering to the subj ect:
1 mg/kg to 3 mg/kg of an anti-PD1 antibody and a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1 × 10⁸ infectious units (IFU) of an MVA virus at about week 9, wherein the MVA virus comprises a nucleotide sequence encoding the amino acid sequence of SEQ ID NO: 1;
1 mg/kg to 3 mg/kg of an anti-PD1 antibody and a vaccine comprising 1 × 10⁸ IFU of the MVA virus at about week 15 and about week 24; and
1 mg/kg to 3 mg/kg of an anti-PD1 antibody and a vaccine comprising 1 × 10⁸ IFU of the MVA virus at about week 36, about week 48, and about week 60.

Embodiment 56. The method of any one of the previous embodiments, wherein the myeloproliferative disease is selected from primary myelofibrosis (MPN), polycythemia vera (PV), essential thrombocythemia (ET), primary myelofibrosis (PFM), secondary myelofibrosis, acute myeloid leukemia (AML), secondary AML, chronic myelogenous leukemia (CML), clonal hematopoiesis of indeterminate potential (CHIP), and chronic myelomonocytic leukemia (CMML).

Embodiment 57. The method of any one of the previous embodiments, wherein the cancer is selected from lung cancer, lymphoid cancer, acute lymphoid leukemia, acute myeloid leukemia, chronic myelogenous leukemia, Burkitt’s lymphoma, Hodgkin’s lymphoma, plasma cell myeloma, biliary tract cancer, bladder cancer, liver cancer, pancreatic cancer, prostate cancer, skin cancer, thyroid cancer, stomach cancer, large intestine cancer, colon cancer, urinary tract cancer, central nervous system cancer, neuroblastoma, kidney cancer, breast cancer, cervical cancer, testicular cancer, and soft tissue cancer.

Embodiment 58. The method of any one of the previous embodiments, wherein the cardiovascular disease is selected from an acute coronary syndrome, an ischemic cerebrovascular disease, an ischemic heart disease, a thrombosis, a venous thromboembolism, a deep vein thrombosis, a pulmonary embolism, a catastrophic intra-abdominal thromboses, a peripheral arterial disease, a hypertension, a heart failure, an atrial fibrillation, a coronary heart disease, an atherosclerosis, and a clonal hematopoiesis.

What is claimed:

1. A method of treating or preventing a myeloproliferative disease, a cancer, or a cardiovascular disease, or inducing an immune response, in a subject having a JAK2V617F substitution and/or a CALR exon 9 mutation, the method comprising administering to the subject a treatment regimen comprising:
two or more vaccines comprising a great ape adenovirus serotype 20 (GAd20) virus that, in turn, comprises a nucleotide sequence encoding the amino acid sequence of SEQ ID NO: 1 and
one or more vaccines comprising a Modified Vaccinia Ankara (MVA) virus that, in turn, comprises a nucleotide sequence encoding the amino acid sequence of SEQ ID NO: 1 to thereby treat or prevent the myeloproliferative disease, cancer, or cardiovascular disease, or induce the immune response.

2. The method of claim 1, further comprising administering the treatment regimen two or more times.

3. The method of claim 1, further comprising administering:
one or more vaccines comprising the GAd20 virus,
one or more vaccines comprising the MVA virus, or
one or more vaccines comprising the GAd20 virus and one or more vaccines comprising the MVA virus.

4. The method of claim 1, wherein each of the vaccines comprising the GAd20 virus comprises about 1 × 10⁹ viral particles (VP) to about 1 × 10¹³ VP of the GAd20 virus.

5. The method of claim 1, wherein each of the vaccines comprising the MVA virus comprises about 1 × 10⁶ infectious units (IFU) to about 1 × 10¹⁰ IFU of the MVA virus.

6. The method of claim 1, further comprising administering the anti-CTLA4 antibody with:
the vaccines comprising the GAd20 virus;
the vaccines comprising the MVA virus; or both.

7. The method of claim 6, comprising administering 0.5 mg/kg to 5 mg/kg of the anti-CTLA4 antibody.

8. The method of claim 1, further comprising administering the anti-PD-1 antibody with:
the vaccines comprising the GAd20 virus;
the vaccines comprising the MVA virus; or both.

9. The method of claim 8, comprising administering 0.5 mg/kg to 5 mg/kg of the anti-PD-1 antibody.

10. The method of claim 1, wherein each of the one or more GAd20 viruses comprise the nucleotide sequence of SEQ ID NO: 2.

11. The method of claim 10, wherein the nucleotide sequence further comprises an N-terminal TCE.

12. The method of claim 1, wherein each of the one or more MVA viruses comprise the nucleotide sequence of SEQ ID NO: 2.

13. The method of claim 12, wherein the nucleotide sequence further comprises an N-terminal TCE.

14. The method of claim 1, comprising administering a vaccine comprising the GAd20 virus at week 0, about week 3, about week 15, and week 18, and administering a vaccine comprising the MVA virus at about week 9, about week 24, about week 36, about week 48, and about week 60.

15. The method of claim 1, wherein the myeloproliferative disease is selected from primary myelofibrosis (MPN), polycythemia vera (PV), essential thrombocythemia (ET), primary myelofibrosis (PFM), secondary myelofibrosis, acute myeloid leukemia (AML), secondary AML, chronic myelogenous leukemia (CML), clonal hematopoiesis of indeterminate potential (CHIP), and chronic myelomonocytic leukemia (CMML).

16. The method of claim 1, wherein the cancer is selected from lung cancer, lymphoid cancer, acute lymphoid leukemia, acute myeloid leukemia, chronic myelogenous leukemia, Burkitt’s lymphoma, Hodgkin’s lymphoma, plasma cell myeloma, biliary tract cancer, bladder cancer, liver cancer, pancreatic cancer, prostate cancer, skin cancer, thyroid cancer, stomach cancer, large intestine cancer, colon cancer, urinary tract cancer, central nervous system cancer, neuroblastoma, kidney cancer, breast cancer, cervical cancer, testicular cancer, and soft tissue cancer.

17. The method of claim 1, wherein the cardiovascular disease is selected from an acute coronary syndrome, an ischemic cerebrovascular disease, an ischemic heart disease, a thrombosis, a venous thromboembolism, a deep vein thrombosis, a pulmonary embolism, a catastrophic intra-abdominal thromboses, a peripheral arterial disease, a hypertension, a heart failure, an atrial fibrillation, a coronary heart disease, an atherosclerosis, and a clonal hematopoiesis.

18. A method of treating or preventing a myeloproliferative disease, a cancer, or a cardiovascular disease, or inducing an immune response, in a subject having a JAK2V617F substitution and/or a CALR exon 9 mutation, the method comprising administering to the subject:
a vaccine comprising 1 × 10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1 × 10⁸ infectious units (IFU) of an MVA virus at about week 9, wherein the MVA virus comprises a nucleotide sequence encoding the amino acid sequence of SEQ ID NO: 1;
a vaccine comprising 1 × 10¹¹ VP of the GAd20 virus at about week 15 and about week 18;
a vaccine comprising 1 × 10⁸ IFU of the MVA virus at about week 24; and
a vaccine comprising 1 × 10⁸ IFU of the MVA virus at about week 36, about week 48, and about week 60.

19. The method of claim 18, further comprising administering 1 mg/kg to 3 mg/kg of an anti-CTLA4 antibody with:
the vaccines comprising the GAd20 virus;
the vaccines comprising the MVA virus; or both.

20. The method of claim 18, further comprising administering 1 mg/kg to 3 mg/kg of an anti-PD-1 antibody with:
the vaccines comprising the GAd20 virus;
the vaccines comprising the MVA virus; or both.

21. The method of claim 18, wherein the GAd20 virus comprises the nucleotide sequence of SEQ ID NO: 2.

22. The method of claim 18, wherein the MVA virus comprises the nucleotide sequence of SEQ ID NO: 2.

23. The method of claim 18, wherein the myeloproliferative disease is selected from primary myelofibrosis (MPN), polycythemia vera (PV), essential thrombocythemia (ET), primary myelofibrosis (PFM), secondary myelofibrosis, acute myeloid leukemia (AML), secondary AML, chronic myelogenous leukemia (CML), clonal hematopoiesis of indeterminate potential (CHIP), and chronic myelomonocytic leukemia (CMML).

24. The method of claim 18, wherein the cancer is selected from lung cancer, lymphoid cancer, acute lymphoid leukemia, acute myeloid leukemia, chronic myelogenous leukemia, Burkitt’s lymphoma, Hodgkin’s lymphoma, plasma cell myeloma, biliary tract cancer, bladder cancer, liver cancer, pancreatic cancer, prostate cancer, skin cancer, thyroid cancer, stomach cancer, large intestine cancer, colon cancer, urinary tract cancer, central nervous system cancer, neuroblastoma, kidney cancer, breast cancer, cervical cancer, testicular cancer, and soft tissue cancer.

25. The method of claim 18, wherein the cardiovascular disease is selected from an acute coronary syndrome, an ischemic cerebrovascular disease, an ischemic heart disease, a thrombosis, a venous thromboembolism, a deep vein thrombosis, a pulmonary embolism, a catastrophic intra-abdominal thromboses, a peripheral arterial disease, a hypertension, a heart failure, an atrial fibrillation, a coronary heart disease, an atherosclerosis, and a clonal hematopoiesis.
